(19) United States
(12) Patent Application Publication
(10) Pub. No.: US 2022/0104801 A1
(43) Pub. Date: Apr. 7, 2022

(54) DRIVER WITH BRAKE SYSTEM FOR ADJUSTING SUTURE TENSION WHILE SECURING A KNOTLESS SUTURE ANCHOR IN A BONE TUNNEL

(71) Applicant: HS West Investments, LLC, Sandy, UT (US)

(72) Inventor: Hugh S. West, JR., Sandy, UT (US)

(21) Appl. No.: 17/490,066

(22) Filed: Sep. 30, 2021

Related U.S. Application Data
(60) Provisional application No. 63/086,584, filed on Oct. 1, 2020.

(51) Int. Cl. A61B 17/04

(52) U.S. Cl. CPC A61B 17/0401; A61B 2017/044; A61B 2017/0427; A61B 2017/0403; A61B 2017/0409; A61B 2017/0453

(57) ABSTRACT

Driver apparatus and methods for adjusting suture tension while driving a suture anchor into a bone tunnel during connective tissue repair surgery, such as hip or shoulder repair. The driver apparatus includes a drive shaft, a driver tip at a distal end for engaging a suture anchor, and a driver handle at a proximal end that includes a manually actuatable suture brake associated or integrated with the handle. The driver apparatus can be used in a method for inserting a suture anchor into a bone tunnel while making adjustments to suture tension by selectively actuating or releasing the suture brake while the suture anchor is being driven into a bone tunnel. The driver apparatus can be adapted for use with single stage push anchors, two-stage push anchors, two-stage threaded anchors, and knotless suture anchors having internal locking means.

### CROSS REFERENCE TO RELATED APPLICATION

This Application claims the benefit of U.S. Provisional Application No. 63/086,584, filed Oct. 1, 2021, which is incorporated by reference in its entirety.

### FIELD OF THE INVENTION

The invention is in the field of suture anchors and drivers, particularly knotless suture anchors and drivers for securing knotless suture anchors into a bone tunnel while adjusting suture tension.

### BACKGROUND

Ligaments, tendons, and other soft tissue that have torn away from a bone can be reattached to the bone using sutures attached to implants commonly referred to as “suture anchors” or “bone anchors”. This repair technique can be used to repair soft tissue or connective tissue, such as the labrum (L) found in a shoulder joint (S), as illustrated in FIGS. 1A-1B, or a hip joint (H), as illustrated in FIGS. 2A-2C.

As illustrated in FIGS. 1A-1B, the shoulder joint (S) is the main joint in a group of structures comprising the shoulder. The shoulder joint (S), or glenohumeral joint, is a ball and socket joint that allows the arm to rotate in a circular fashion or to hinge out and up away from the body. The labrum (L) is a piece of fibrocartilage attached to the rim of the shoulder socket, or glenoid (G), which helps keep the humeral head (HH) of the humerus (HU) in place.

As illustrated in FIGS. 2A-2C, in a hip joint (H), the labrum (L) surrounds the acetabulum (A), or hip socket, which houses the femoral head (FH) of the femur (F), and facilitates and defines the normal hip range of motion, providing both support and flexibility to the joint. When the labrum (L) is torn, this is called a labral tear (LT). Labral tears (LT) may result from injury and/or aging.

As illustrated in FIGS. 1A-1B for shoulder joint repair and FIGS. 2A-2C for hip joint repair, sutures 1 are attached to or looped around the torn labrum L. The free ends of sutures 1 are threaded through a bone anchor 2 and passed out of the body. A driver is used to drive the bone anchor 2 into a pre-drilled bone tunnel in order to “lock” the sutures in place relative to the bone and hold damaged connective tissue against the bone to promote healing through formation of scar tissue.

To ensure proper healing and rejoining of soft tissue to bone, the sutures attached to the labrum should be properly tensioned in order for the labrum to be slightly compressed and securely held against the bone. If the sutures are too loose, the labrum (L) can pull away from the bone and fail to properly rejoin with the bone, which may require additional surgery to correct the problem. If the sutures are too tight, they can cut into and further damage the already damaged labrum, which may require additional surgery to correct and/or cause long-term or permanent labral damage.

Anchors used to secure a damaged labrum to bone typically include an eyelet through which sutures attached to the labrum are slidably threaded and tensioned and are driven into a pre-drilled bone tunnel using a driver. A typical driver includes a handle at the proximal end and a driver tip at the distal end attached to a suture anchor. During initial placement of the suture anchor into a bone tunnel, the surgeon grips the driver handle with one hand to position the driver tip and anchor at the opening of the bone tunnel. The surgeon uses the other hand to pull on the free suture ends and apply tension to the sutures. The eyelet transmits the applied suture tension to the labrum, pulling it toward the eyelet and the bone with essentially the same tensile force as the tension applied to the free suture ends. The suture anchor is then driven into the bone tunnel using the driver to fix the suture tension that will hold the labrum against the bone. In the case of a push anchor, the surgeon typically strikes the driver handle several times (e.g., 8-12 times) using a mallet. An endoscope can be used to visually determine whether the sutures and labrum are properly tensioned against the bone.

There are several things that can go wrong and prevent the sutures and labrum from being properly tensioned. The initially applied tension may not be optimal to ensure proper healing. If suture tension is too low, the surgeon must further tension the sutures before the anchor is fully locked in place within the bone tunnel. If suture tension is too high, the surgeon must loosen the sutures before the anchor is fully locked in place. Either adjustment can be difficult and inexact and, if done improperly, can require the anchor to be removed and reinserted to properly reset tension.

Much of the problem results from inadequately designed drivers conventional in the art. Some drivers require continuous manual application of tension while the anchor is being driven into the bone tunnel. In such case, a third hand of an assistant may be required to maintain suture tension while the surgeon uses one hand to grip the driver handle and the other to drive the suture anchor into the bone tunnel, such as by striking the driver handle with a mallet. If the assistant fails to maintain proper tension, or if the surgeon wants more control over the tensioning process, the surgeon must release the mallet, grip and apply tension to the free suture ends using the mallet hand, hand the free sutures ends back to the assistant, grab the mallet, and resume striking the driver handle with the mallet to continue driving the anchor into place. This procedure may need to be repeated several times to achieve optimal tension, causing a lack of continuity between driving the anchor into the bone tunnel and maintaining proper suture tension.

Some drivers are equipped with a cleat or other locking mechanism to which tensioned suture ends can be attached to maintain or increase suture tension while the anchor is being driven into the bone tunnel. If tension is either too low or too high, the surgeon or assistant may have to adjust suture tension between mallet strikes by releasing the free suture ends from the cleat, manually increasing or decreasing suture tension, and reattaching the suture ends to the cleat, after which the surgeon can resume driving the suture anchor into the bone tunnel. Again, this causes lack of continuity between driving the anchor into the bone tunnel and maintaining proper suture tension.

Other drivers are equipped with a clutch mechanism that applies a continuous force to the free suture ends in order to maintain constant tension on the sutures as the anchor is driven into the bone tunnel. This type of driver relies on the assumption that forces applied by the clutch mechanism will cause correct tensioning of the sutures. A major flaw is that forces applied by the clutch mechanism to the sutures are preset and cannot be increased or decreased while driving the suture anchor into the bone tunnel. Clutches typically rely on the material properties of elastomeric clutch arms or metal springs attached to a clutch arm or plate to maintain a fixed pressure on the sutures. However, it is unlikely that the clutch will fortuitously apply the exact amount of pressure required to apply and maintain optimal suture tension for a particular labral repair. Moreover, there may not be an optimal suture tension that applies across all labral repairs. Different patients have labra of different sizes and shapes that may require different suture tensions to properly seat each labrum against bone. Also, different types of tears may require different suture tensions. The inability to adjust suture tension using clutch mechanisms greatly reduces their utility.

Still other drivers may permit a surgeon to increase suture tension using a ratchet mechanism in the handle of a driver. A problem with using a ratchet mechanism to increase suture tension is that it provides no tactile feel of actual suture tension. In addition, the ratcheting process is usually different than and out of sync with the process of driving the suture anchor into the bone tunnel, which often leads to poor control over suture tension.

### SUMMARY

Disclosed are driver apparatus and methods for adjusting suture tension while driving a suture anchor into a bone tunnel during connective tissue repair surgery, such as hip or shoulder repair. The driver apparatus includes a drive shaft, a driver tip at a distal end for engaging a suture anchor, and a driver handle at a proximal end that includes a manually actuatable suture brake associated or integrated with the handle. The driver apparatus can be used in a method for inserting a suture anchor into a bone tunnel while making real-time adjustments to suture tension by selectively actuating or releasing the suture brake while the suture anchor is being driven into a bone tunnel.

The manually actuatable suture brake permits a surgeon to make adjustments to suture tension while driving in the suture anchor using just two hands: one to hold the driver handle and selectively actuate or release the suture brake and the other to drive the suture anchor into the bone tunnel, such as by gripping a mallet and striking the driver with the mallet in the case of a push anchor, or rotating a knob in the case of a threaded anchor. By selectively actuating and releasing the suture brake with the handle gripping hand, while driving the suture anchor into the bone tunnel using the other hand, the surgeon can fine tune suture tension in one continuous action using just two hands, e.g., without letting go of the driver or mallet to readjust suture tension and/or without requiring assistance from a third hand.

During insertion of a suture anchor into a bone tunnel, the free ends of sutures attached to soft tissue, such as a labrum, are associated with the suture brake of the driver handle. When actuated, the suture brake applies force to the free suture ends and prevents longitudinal movement of the free suture ends relative to the driver. Releasing the suture brake permits longitudinal movement of the free suture ends relative to the driver. By selectively braking or releasing the free suture ends while driving the suture anchor into a bone tunnel, the surgeon can selectively increase, decrease, or maintain suture tension using just two hands in one continuous action.

In some embodiments, the driver apparatus is adapted for use with a single stage push anchor that includes an eyelet through which sutures attached to soft tissue are threaded. With the anchor attached to the driver tip, the surgeon grips the driver handle with one hand, places the distal tip of the anchor at the opening of a bone tunnel, and, using the other hand, pulls the free suture ends to take up slack, apply an initial suture tension, and pull the soft tissue toward the bone. This can be done before, during or after associating the free suture ends with the driver handle and suture brake. While gripping the driver handle and selectively actuating or releasing the suture brake with one hand, the surgeon can strike the driver (e.g., driver handle) with a mallet using the other hand to progressively drive the anchor into the bone tunnel using several (e.g., 8 to 12) mallet strikes. Actuating the suture brake while driving the anchor into the bone tunnel prevents slippage of sutures through the eyelet, thereby maintaining a substantially fixed suture length between the soft tissue and eyelet and progressively increasing suture tension with each mallet strike. Conversely, releasing the suture brake permits slippage of sutures through the eyelet, which permits the anchor and eyelet to be driven further into the bone tunnel without significantly increasing suture tension. Depending on the depth of the suture anchor, releasing the suture brake may relax suture tension. Suture tension can then be selectively maintained or increased by selectively actuating or releasing the suture brake as the suture anchor is driven more deeply into the bone tunnel.

By selectively actuating and releasing the suture brake, the surgeon can increase, maintain, or reduce suture tension as desired to obtain optimal tension while driving the push anchor into the bone tunnel. For example, optimal suture tension may be achieved when the soft tissue has been compressed to a certain degree against the bone, but not overly compressed, which can be observed using an endoscope. When suture tension is insufficient, the surgeon can actuate the suture brake while driving the suture anchor into the bone tunnel to increase suture tension. When optimal suture tension has been attained, the surgeon can release the suture brake and continue driving the suture anchor all the way into the bone tunnel to maintain and fix the optimal suture tension.

In other embodiments, the driver apparatus can be adapted for use with other types of suture anchors, such as two-stage push anchors, two-stage threaded anchors, or knotless suture anchors having internal locking means. In this way, the inventive driver apparatus is versatile and can be used with a range of different suture anchors while permitting adjustments to suture tension using two hands, e.g., without having to let go of the driver handle and/or mallet to adjust suture tension and/or without the assistance of a third hand.

These and other advantages and features of the invention will become more fully apparent from the following description and appended claims, or may be learned by the practice of the invention as set forth hereinafter.

### BRIEF DESCRIPTION OF THE DRAWINGS

In order to describe the manner in which at least some of the advantages and features of the invention may be obtained, a more particular description of embodiments of the invention will be rendered by reference to specific embodiments thereof which are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings.

FIGS. 1A-1B illustrate the anatomy of a shoulder joint with a torn labrum and subsequent repair of the torn labrum.

FIGS. 2A-2C illustrate the anatomy of a hip joint with a torn labrum and subsequent repair of the torn labrum.

FIGS. 3A-3E illustrate a driver apparatus of the invention adapted for use with a single stage push anchor and having a handle equipped with a manually actuatable suture brake.

FIGS. 4A-4G illustrate a driver apparatus adapted for use with a two-stage push anchor and having a handle equipped with a manually actuatable suture brake.

FIGS. 5A-5G illustrate an embodiment of a driver apparatus adapted for use with a two-stage threaded anchor and having a handle equipped with a manually actuatable suture brake.

FIGS. 5AA-5GG illustrate another embodiment of a driver apparatus adapted for use with a two-stage threaded anchor and having a handle equipped with a manually actuatable suture brake.

FIGS. 5AAA-5GGG illustrate another yet embodiment of a driver apparatus adapted for use with a two-stage threaded anchor and having a handle equipped with a manually actuatable suture brake.

FIGS. 6A-6AA are closeup views that illustrate a single stage push anchor.

FIGS. 6B-6BB are closeup views that illustrate a two-stage push anchor.

FIGS. 6C-6CC are closeup views that illustrate a two-stage threaded anchor.

FIGS. 6D-6DDD are closeup views that illustrate a knotless suture anchor with internal locking means.

FIG. 7A is an exploded view of a driver for use with a single stage push anchor, such as in FIGS. 3A-3D and 6A-6AA, having a handle equipped with a manually actuatable suture brake for selectively engaging free suture ends to adjust suture tension while driving the single stage push anchor into a bone tunnel.

FIG. 7B is an assembled view of the driver of FIG. 7A.

FIG. 7C depicts the driver of FIG. 7B being used to position a single stage push anchor at a bone tunnel opening, with one hand gripping the driver handle and another hand gripping and applying initial tension to the free suture ends.

FIG. 7D depicts the driver with one hand gripping the driver handle and selectively actuating or releasing the suture brake to increase, decrease, or maintain suture tension as the push anchor is being driven into the bone tunnel using a mallet gripped by the other hand.

FIG. 7E depict the driver with the push anchor driven fully into the bone tunnel in order to lock to the suture in place with a desired tension.

FIG. 7F depict the driver removed and the free suture ends being cut.

FIGS. 8A and 8B depict another embodiment of a driver with a handle equipped with a squeezable elastic suture brake.

FIGS. 9A and 9B depict another embodiment of a driver apparatus with handle equipped with a suture brake lever.

### DETAILED DESCRIPTION OF SOME EXAMPLE EMBODIMENTS

Embodiments of the present invention generally relate to apparatus and methods for adjusting suture tension during orthopedic procedures, such as shoulder or hip repair surgeries (e.g., for labral repair). More particularly, the invention relates to driver apparatus and methods for securing a suture anchor to a bone during a connective tissue repair procedure that facilitate real time adjustments to suture tension.

An example driver apparatus is equipped with a selectively actuatable and releasable suture brake mechanism for adjusting suture tension during insertion of a bone anchor in a connective tissue repair procedure. Such repair procedure can involve attaching connective tissue in the hip or shoulder repair surgery using suture anchors. The driver apparatus comprises a drive shaft, a driver tip at the distal end for engaging a suture anchor, and a proximal handle portion equipped with a suture brake that can be selectively actuated or released to engage or disengage free suture ends of sutures attached to soft tissue. The handle and brake mechanism can be configured to allow a surgeon to have single-handed control over tension applied to sutures throughout insertion of the bone anchor into a bone tunnel.

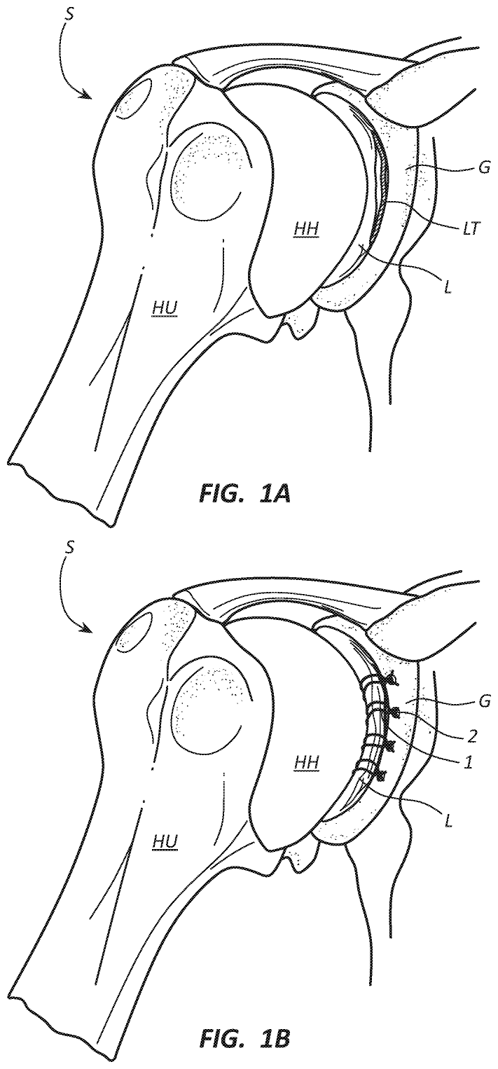

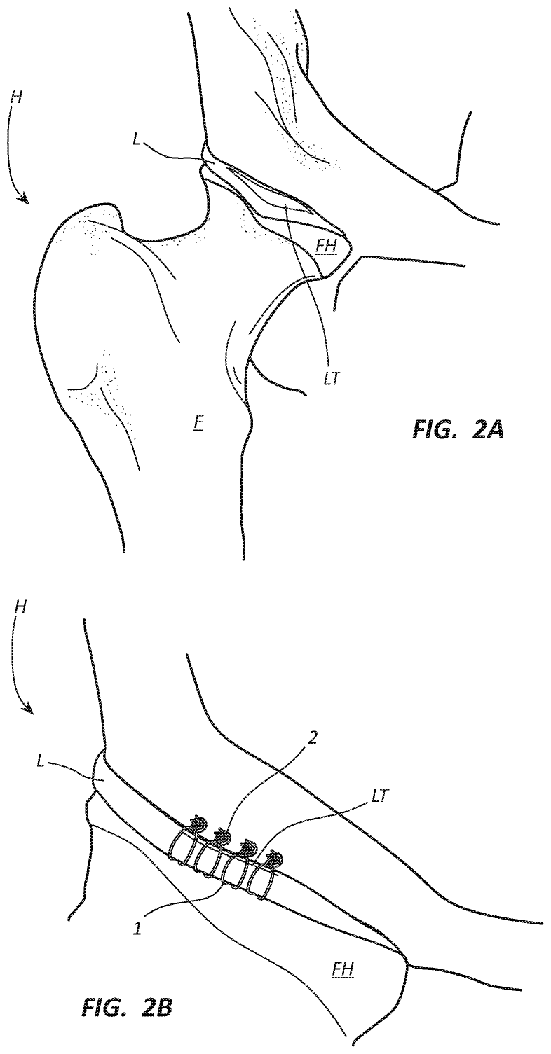

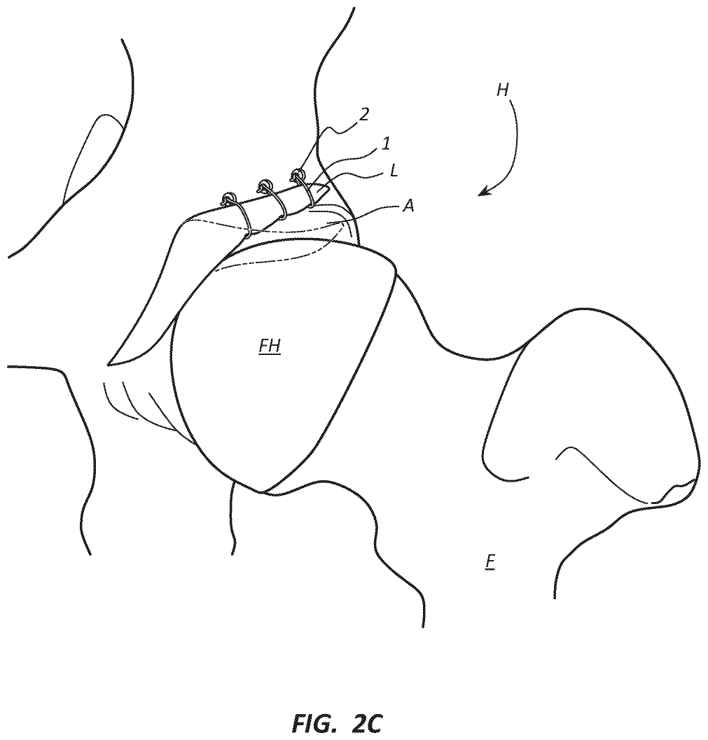

The Driver apparatus according to the invention can be used to adjust suture tension while securing a bone anchor to a bone. For example, the driver apparatus can be used to repair a labrum (L) found in a shoulder joint (S). As illustrated in FIGS. 1A-1B for shoulder joint (S) repair and FIGS. 2A-2C for hip joint (H) repair, sutures 1 are attached to or looped around the torn labrum L. The free ends of sutures 1 are threaded through a bone anchor 2 and passed out of the body, and a driver is used to drive the bone anchor 2 into a pre-drilled bone tunnel in order to lock the sutures in place and hold damaged connective tissue against the bone with desired force to promote healing. To ensure proper healing and rejoining of soft tissue to bone, the sutures attached to soft tissue can be tensioned using the disclosed driver apparatus in order for the soft tissue to be properly compressed and securely held against the bone.

Driver apparatuses and methods according to the invention can be adapted for use with a variety of suture anchors, including single stage push anchors, two-stage push anchors, two-stage threaded anchors, and knotless suture anchors having internal locking means. The inventive driver apparatuses and methods are therefore versatile and can be used with a range of different suture anchors while permitting adjustments to suture tension using two hands, e.g., without having to let go of the driver handle and/or mallet to adjust suture tension and/or without the assistance of a third hand.

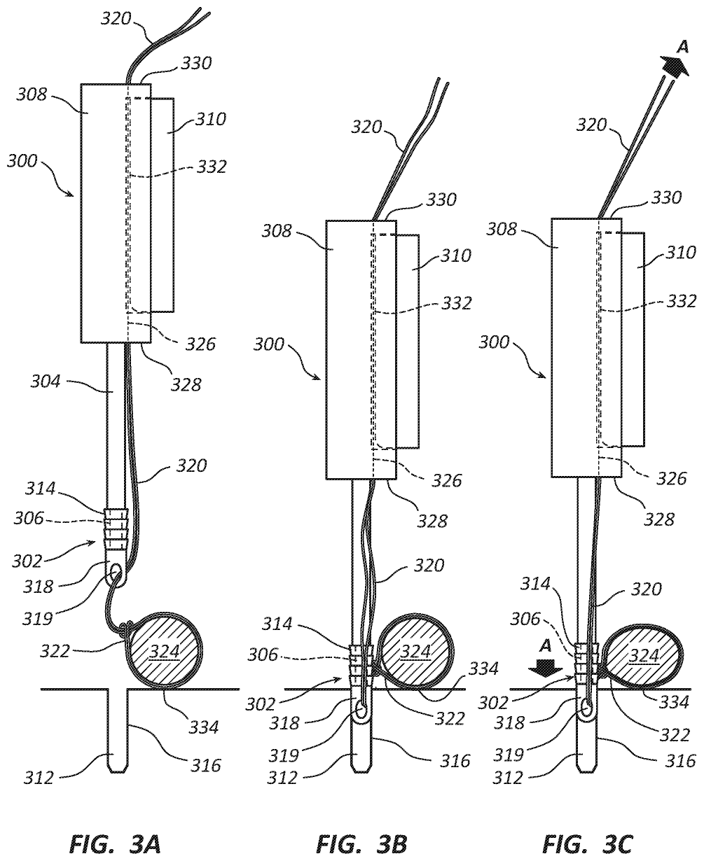

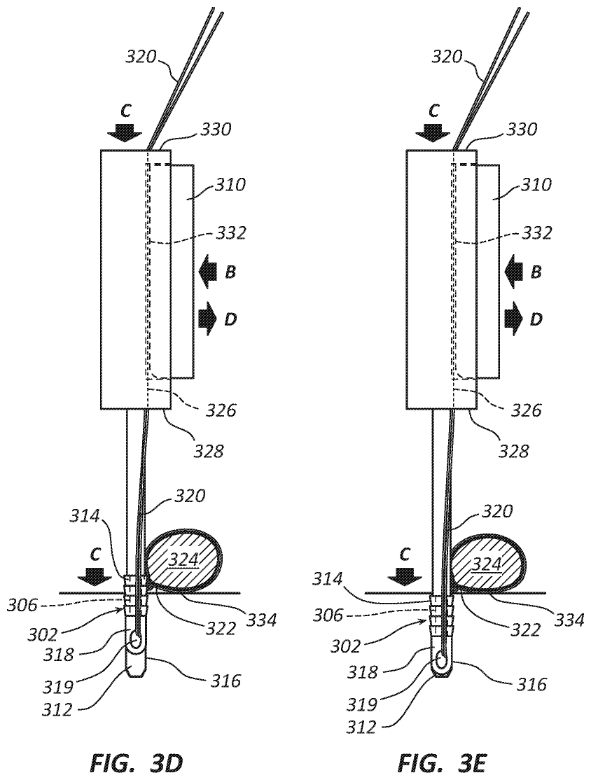

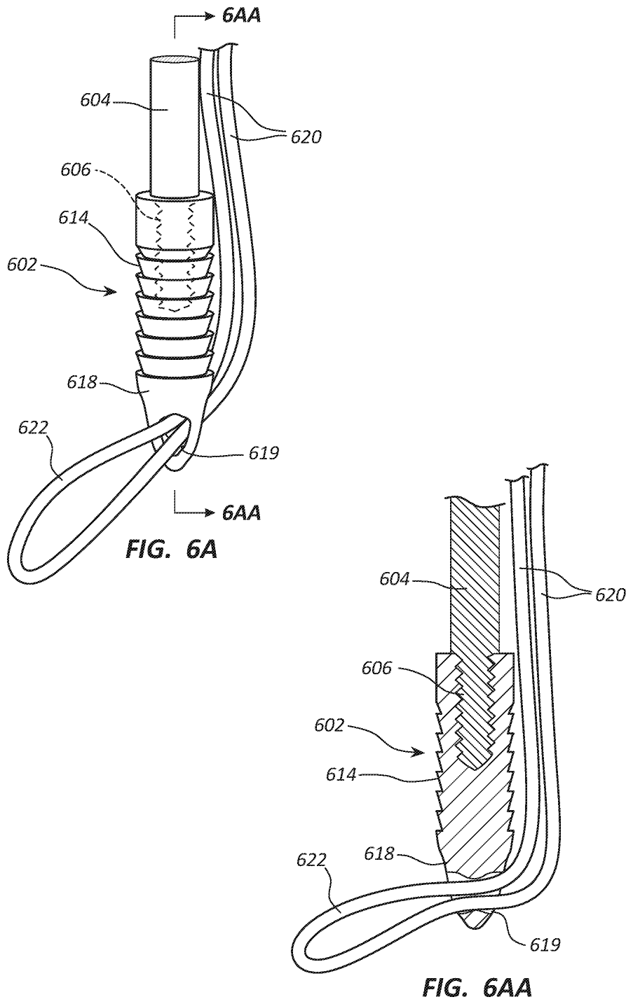

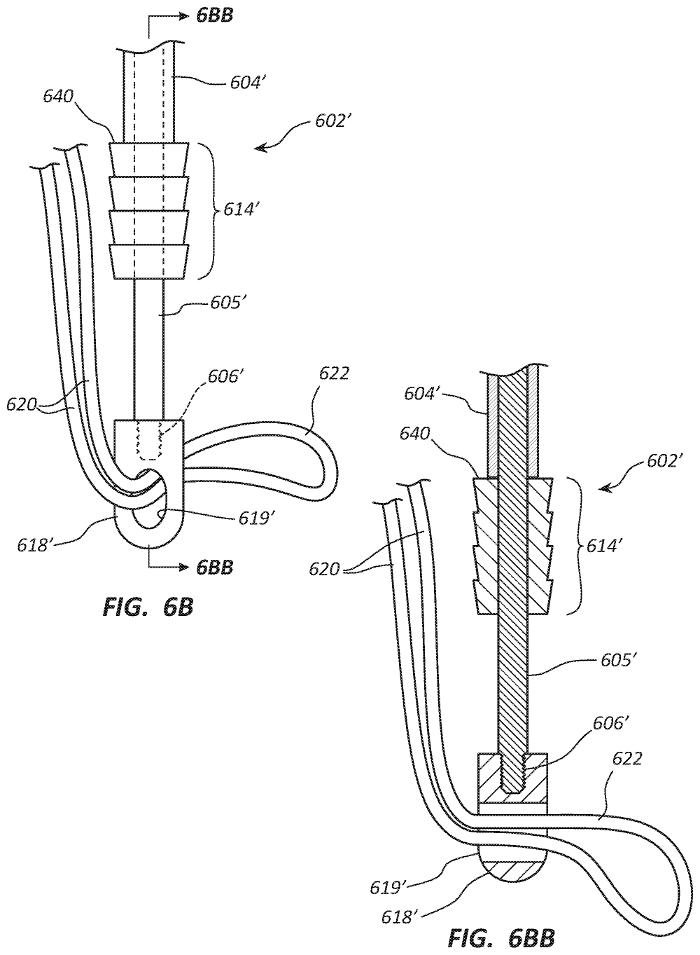

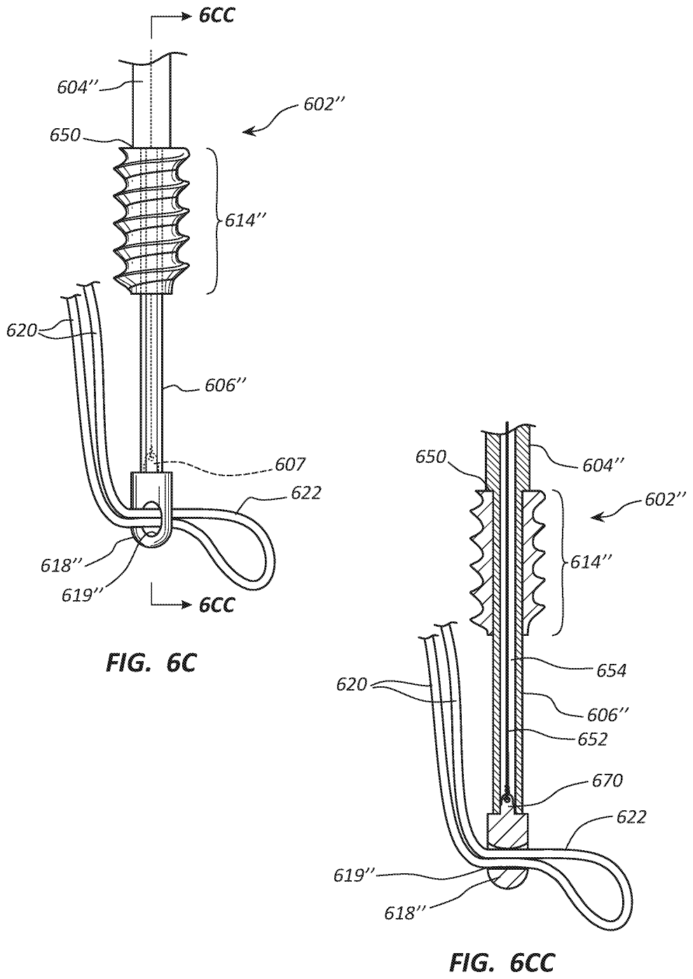

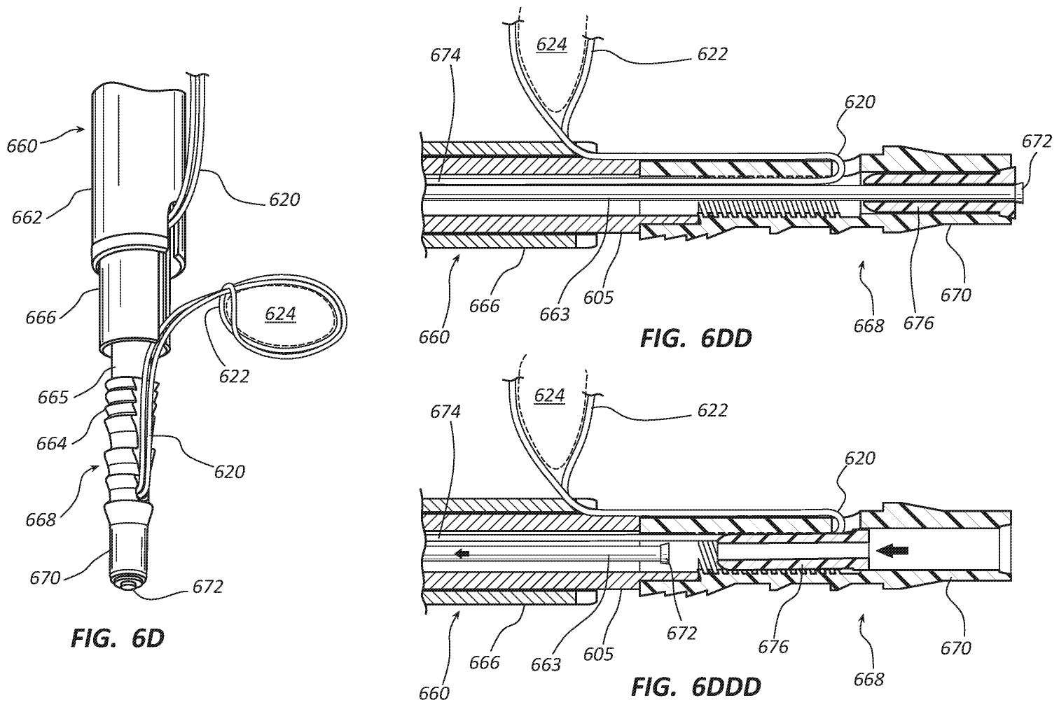

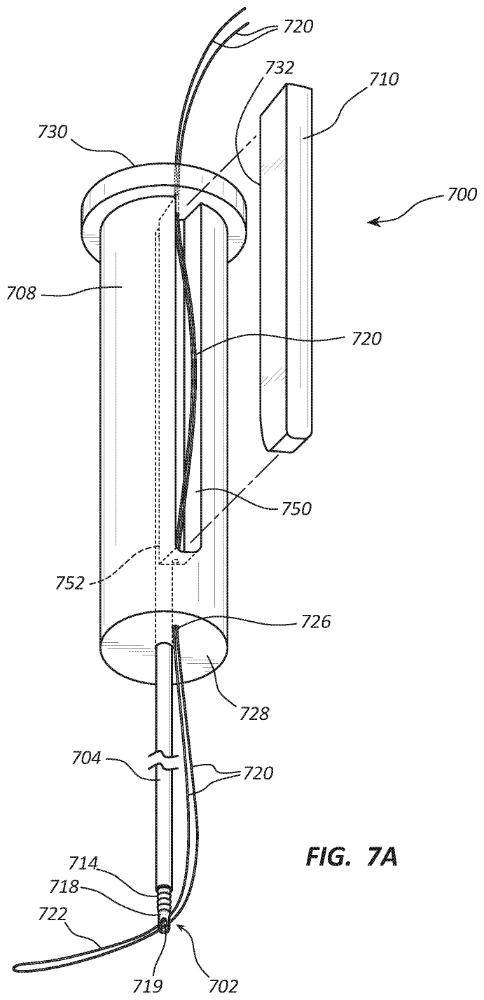

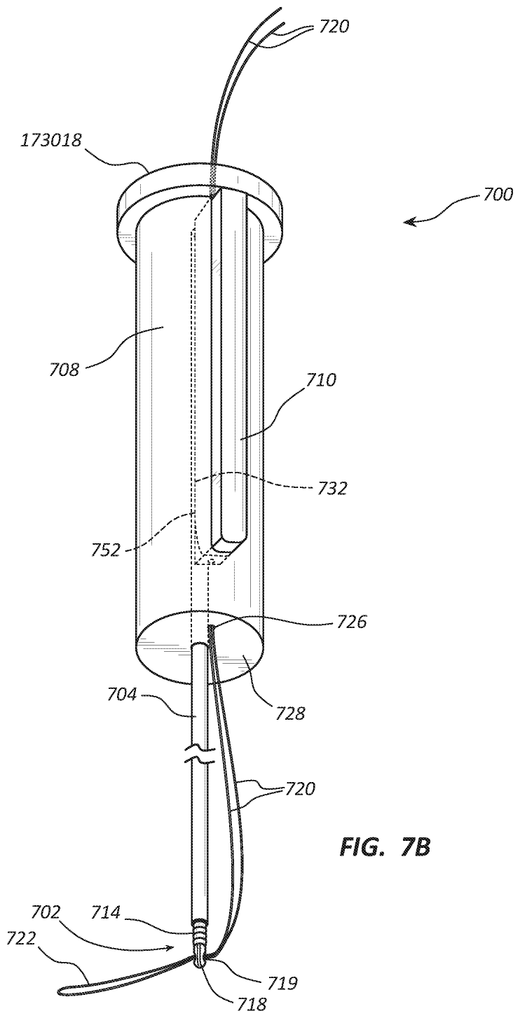

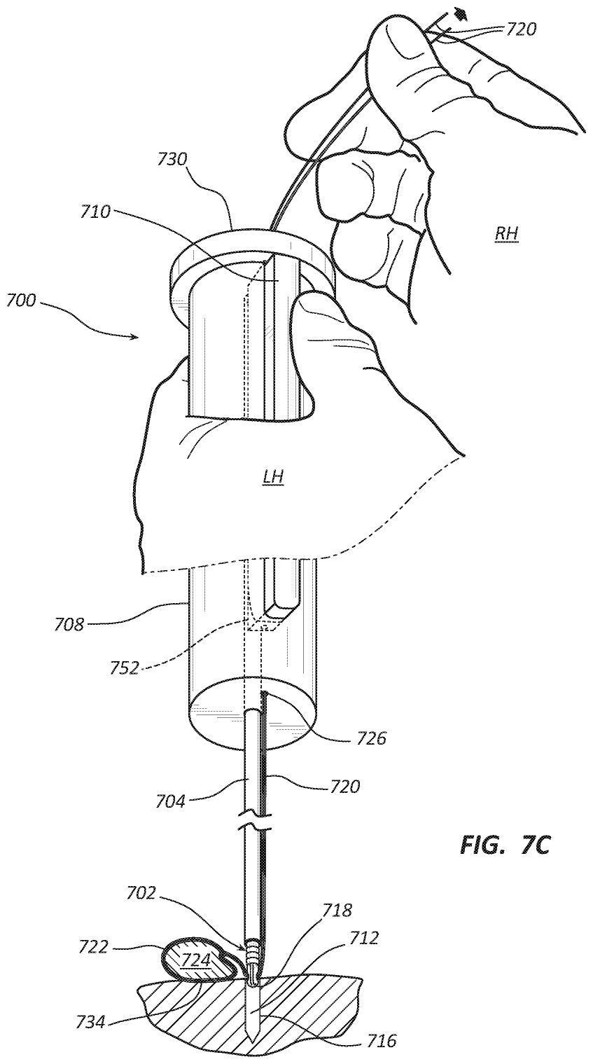

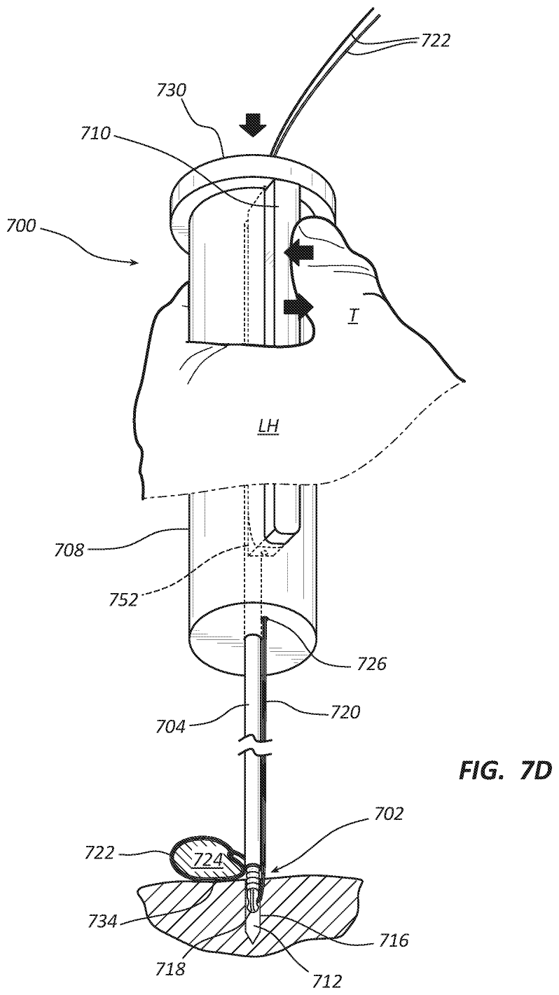

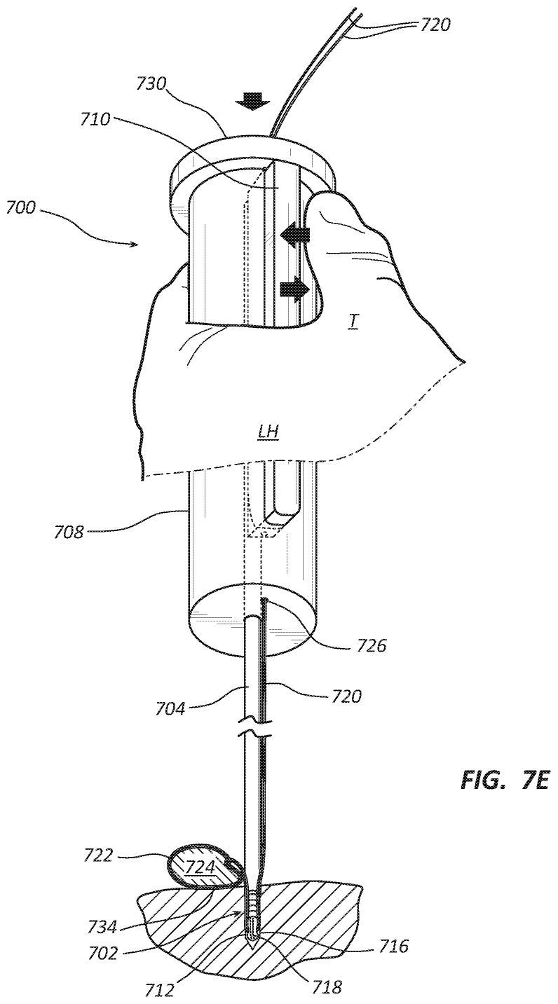

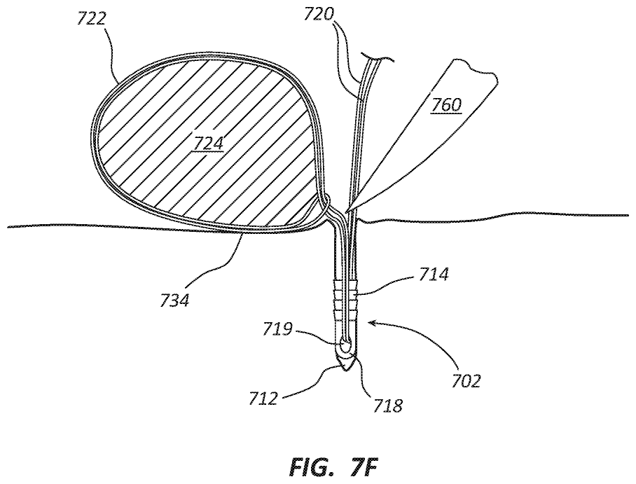

FIGS. 3A-3E illustrate an embodiment of a driver apparatus 300 adapted for use with a single stage push anchor 302 (see FIGS. 6A-6AA). A similar driver apparatus 700 is illustrated in FIGS. 7A-7F and discussed below. Driver apparatus 300 includes a drive shaft 304, a driver tip 306 at a distal end for engaging suture anchor 302, a driver handle 308 at or near a proximal end, and a manually actuatable suture brake 310 associated with driver handle 308. Suture brake 310 is configured to be selectively actuated and released using the same hand that grips driver handle 308, while using the other hand to drive suture anchor 302 into a bone tunnel 312. As illustrated, handle 308 and suture brake 310 can be formed and function as a single unit.

Single stage push anchor 302 includes a barbed portion 314 with barbs configured to engage and obtain purchase with an inner wall 316 of bone tunnel 312 and prevent or resist pullout once installed and an eyelet portion 318 that includes a hole 319 through which sutures 322 attached to soft tissue 324 can be threaded. Push anchor 302 can be attached to driver tip 306 using any known attachment means, such as a friction fit, slidable fit, or threaded fit, such as is illustrated in FIGS. 6A-6AA, in which a threaded tip 606 is received within a threaded socket of push anchor 602. In a friction fit or slidable fit configuration, driver tip 306 can have a non-threaded tip with a cross-sectional shape (e.g., circular, rectangular, pentagonal, hexagonal, or star-shaped) that fits into a correspondingly-shaped socket at a top portion of push anchor 302.

Driver handle 308 includes a suture passageway 326 for receiving free suture ends 320 of suture(s) 322. In the illustrated embodiment, passageway 326 extends between a distal end 328 and a proximal end 330 of handle 308, although handle 308 can be configured so that passageway 326 opens and/or exits at one or more other locations. Handle 308 may include a side slot (not shown) that permits quick placement of free suture ends 320 within passageway 326. Suture passageway 326 and suture brake 310 are configured such that manual application of pressure to suture brake 310 by the hand gripping driver handle 308 causes a braking surface 332 to press against and engage free suture ends 320 with sufficient force to substantially prevent or minimize longitudinal movement relative to driver handle 308. As will be discussed relative to FIGS. 3B-3E, selectively actuating or releasing suture brake 310 permits a surgeon to drive single stage push anchor 302 into bone tunnel 312, such as by striking proximal end 330 of handle LU 308 with a mallet (not shown), while selectively increasing, decreasing, or maintaining tension applied by suture(s) 322 to soft tissue 324.

As illustrated in FIG. 3A, suture(s) 322 are initially threaded through, attached to, and/or wrapped around and then tied to soft tissue 324, which can be a torn labrum of a shoulder or hip. Free suture ends 320 are threaded through eyelet hole 319 of single stage push anchor 302, which is releasably attached to driver tip 306, and positioned within passageway 326 of driver handle 308. In some embodiments, suture brake 310 may be temporarily removed, retracted, or otherwise positioned to facilitate placement of free suture ends 320 within passageway 326. With handle 308, brake 310, and free suture ends 320 properly assembled, driver apparatus 300 is ready for use in driving single stage push anchor 302 into bone tunnel 312 while selectively adjusting suture tension.

As illustrated in FIG. 3B, a first step involves placing eyelet portion 318 of single stage push anchor 302 into the opening of bone tunnel 312. This can be done by gripping handle 308 with one hand and positioning eyelet portion 318 at the opening of bone tunnel 312. At this stage, free suture ends 320 will typically be loose with some slack. Slack can be taken up by pulling free suture ends 320 with the other hand.

As illustrated in FIG. 3C, a second step involves applying a tensile force A to the portion free suture ends 320 extending from driver handle 308 to take up suture slack and cause free suture ends 320 to become taught. This can be done with two hands by gripping driver handle 308 with one hand and pulling on free suture ends 320 with the other hand. Because free suture ends 320 of suture(s) 322 are slidably threaded through eyelet hole 319, the upward tensile force A applied to free suture ends 320 extending from driver handle 308 is transmitted by eyelet 318 to the portion of suture(s) 322 attached to soft tissue 324. This results in a downward tensile force A of essentially equal magnitude, which pulls soft tissue 324 toward bone surface 334 adjacent to bone tunnel 312 (assuming minimal friction or other impediment to the equalization of tensile force A throughout suture(s) 322).

Depending on the magnitude of tensile force A and the compressibility of soft tissue 324, downward tensile force A may be sufficient to cause deformation and compression of soft tissue 324, resulting in an initial suture tension. In some cases, the initial suture tension may already be the optimal tension. Releasing suture brake 310 when driving push anchor 302 into bone hole 312 permits suture(s) 322 to slide through eyelet to increase the length of suture(s) 322 between eyelet 318 and soft tissue 324 to maintain suture tension or, depending on whether and/or how much the anchor barbs have gained purchase with adjacent bone, reduce suture tension. In other cases, it may be necessary to further increase suture tension. This can be accomplished by driving single stage push anchor 302 into bone tunnel 312 using driver apparatus 300 while selectively actuating suture brake 310.

As illustrated in FIGS. 3D and 3E, push anchor 302 can be driven into bone tunnel 312 by applying a downward force C to handle 308, which is transmitted through drive shaft 304 to push anchor 302. Downward force C can be applied using any appropriate means, such as by striking driver handle 308 with a mallet (not shown). Where handle 308 and drive shaft 304 are rigid, the downward force C applied to handle 308 and push anchor 302 are essentially the same. By selectively actuating or releasing suture brake 310 (represented by manual squeezing force B and release action D) in order for braking surface 332 to engage or release free suture ends 320, the surgeon can selectively increase, maintain, or reduce suture tension while driving push anchor 302 into bone tunnel 312 using just two hands: one to grip driver handle 308 and selectively manipulate suture brake 310 and the other to grip a mallet to strike handle 308 with a downward force C. Assistance from a third hand is not required. This provides the surgeon with essentially total control over the acts of driving in push anchor 302 and adjusting tension on suture(s) 322 and soft tissue 324 without outside assistance. In this way, driving in single stage push anchor 302 and adjusting suture tension can be performed in one continuous action without the usual interruptions required when using conventional drivers.

FIG. 3D illustrates a configuration where push anchor 302 has been driven partially into bone tunnel 312 using several taps (e.g., 4 to 6) of a mallet. Part of barbed portion 314 of push anchor 302 is shown engaging inner wall 316 of bone tunnel 312. Each tap of the mallet drives anchor 302 deeper into bone tunnel 312, causing more of barbed portion 314 to engage bone tunnel wall 316 to gain purchase and incrementally increasing the distance between eyelet portion 318 of push anchor 302 and soft tissue 324. If brake 310 is actuated, the increased distance between eyelet portion 318 and soft tissue 324, coupled with constant suture length between eyelet portion 318 and soft tissue 324, increases suture tension. If brake 310 is released, free ends 320 of suture(s) 322 can slide through eyelet hole 319 to increase the length of suture(s) 322 between eyelet portion 318 and soft tissue 324 as the distance between eyelet portion 318 and soft tissue 324 is increased, which maintains or reduces suture tension depending on how far push anchor 302 has advanced into bone tunnel 312.

FIG. 3E illustrates a final configuration where push anchor 302 has been driven all the way into bone tunnel 312 using several additional taps (e.g., 4 to 6) of the mallet. In the final configuration, all of barbed portion 314 of push anchor 302 engages bone tunnel wall 316, preventing or resisting pullout of anchor 302 from bone tunnel 312 and achieving maximum distance between eyelet portion 318 of push anchor 302 and soft tissue 324. This locks suture(s) 322 within bone tunnel 312 at a final suture tension and holds soft tissue 324 against bone surface 334 with a final force.

During the process of driving in push anchor 302, the surgeon can selectively actuate or release suture brake 310 to adjust suture tension. Actuating suture brake 310 prevents longitudinal movement of free suture ends 320 relative to driver handle 308. This, in turn, maintains an essentially constant length of suture(s) 322 between eyelet portion 318 and soft tissue 324 even as each mallet tap incrementally drives anchor 302 deeper into bone tunnel 312 and increases the distance between eyelet portion 318 and soft tissue 324. The downward pull of anchor 302 and suture(s) 322 on soft tissue 324 by driving force C increases suture tension and pulls soft tissue 324 with greater force against bone surface 334. By the combined action of driving anchor 302 deeper into bone tunnel 312 while actuating suture brake 310, the surgeon can incrementally increase suture tension with each mallet tap.

Conversely, releasing suture brake 310 permits free suture ends 320 to move longitudinally relative to driver handle 308 and slide through eyelet hole 319. This allows the length of suture(s) 322 between eyelet portion 318 and soft tissue 324 to increase as push anchor 302 is driven into bone tunnel 312. When free suture ends 320 are allowed to slide through eyelet hole 319, increasing the distance between eyelet portion 318 and soft tissue 324 with each mallet tap does not significantly increase suture tension. In this way, the surgeon can maintain essentially constant suture tension while continuing to drive suture anchor 302 deeper into bone tunnel 312. Moreover, if suture tension is excessively high when initially driving anchor 302 into bone tunnel 312, and barbed portion 314 has not engaged inner wall 316 of tunnel wall 312 with sufficient force to prevent pullout, it may be possible to reduce suture tension by releasing suture brake 310 and permitting sutures 322 to release excess tension, optionally while backing out suture anchor 302 from bone tunnel 312.

In summary, FIGS. 3A-3E illustrate how driver apparatus 300 facilitates fine, real time adjustments to suture tension while driving a single stage push anchor 302 into a bone tunnel 312. By selectively increasing or maintaining suture tension by selective actuating or releasing suture brake 310 while driving push anchor 302 into bone tunnel 312, the surgeon can adjust and achieve optimal suture tension using only two hands. Because the process of driving push anchor 302 into bone tunnel 312 typically requires several (e.g., 8-12) mallet taps, the surgeon has several opportunities to adjust and fine tune suture tension and readily achieve optimal suture tension in one continuous action, without having to interrupt the process, and without requiring assistance from a third hand.

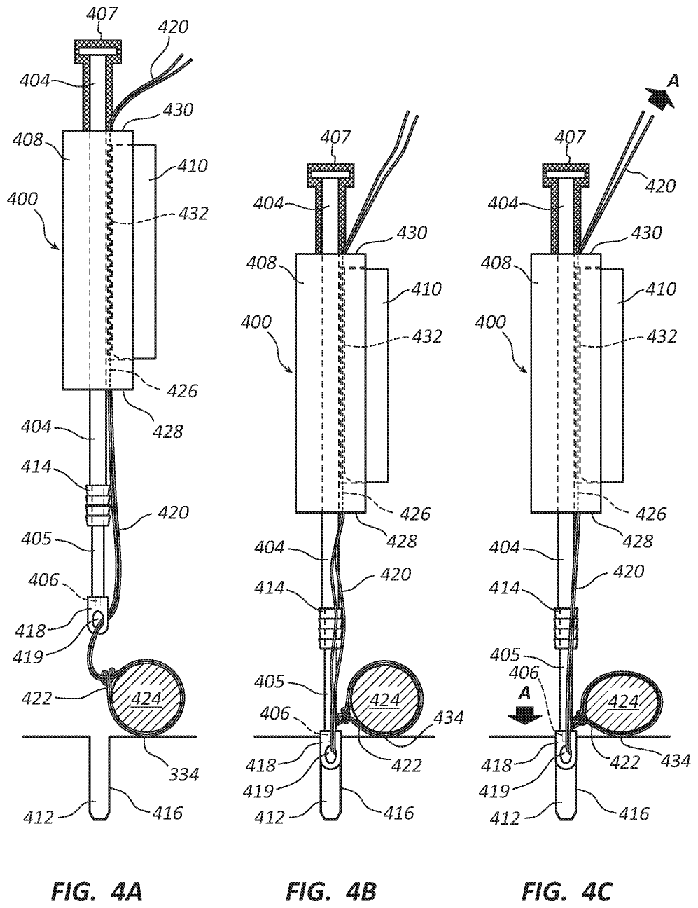

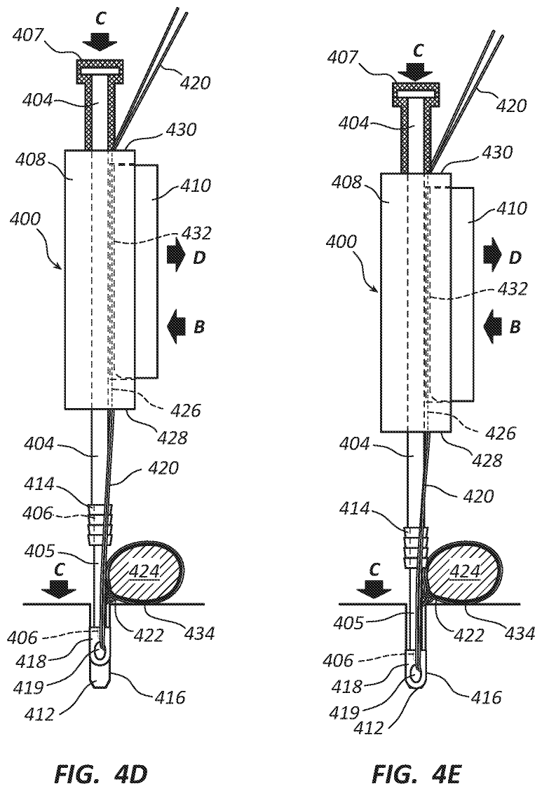

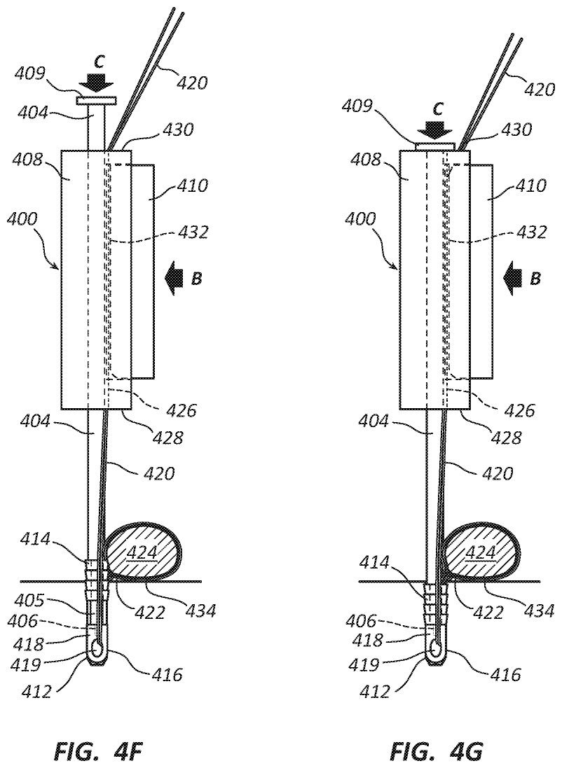

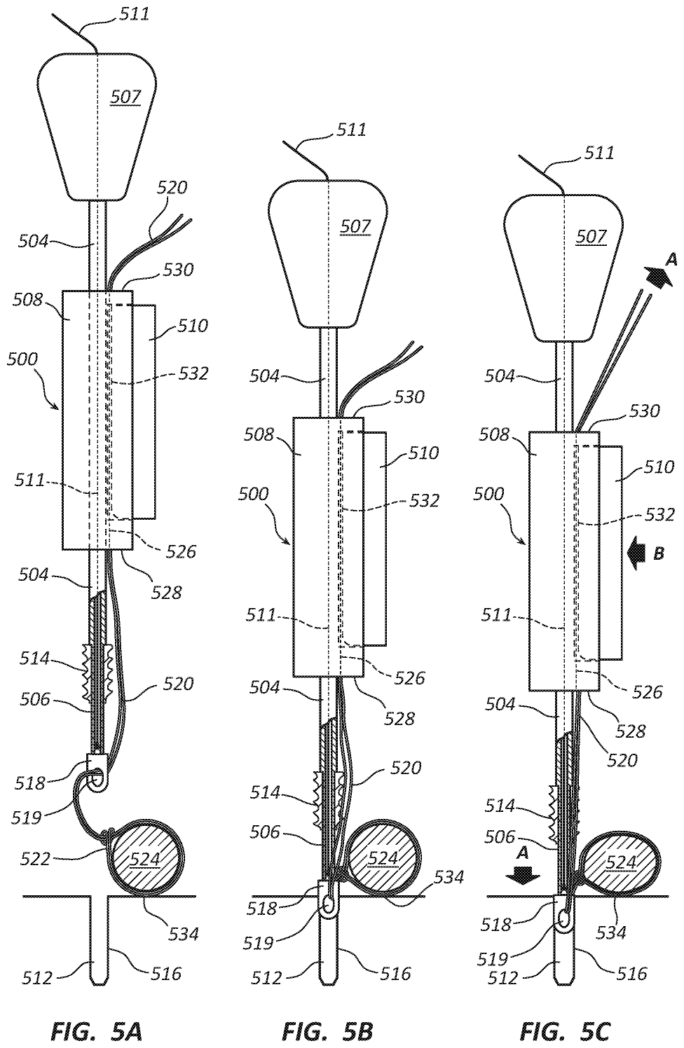

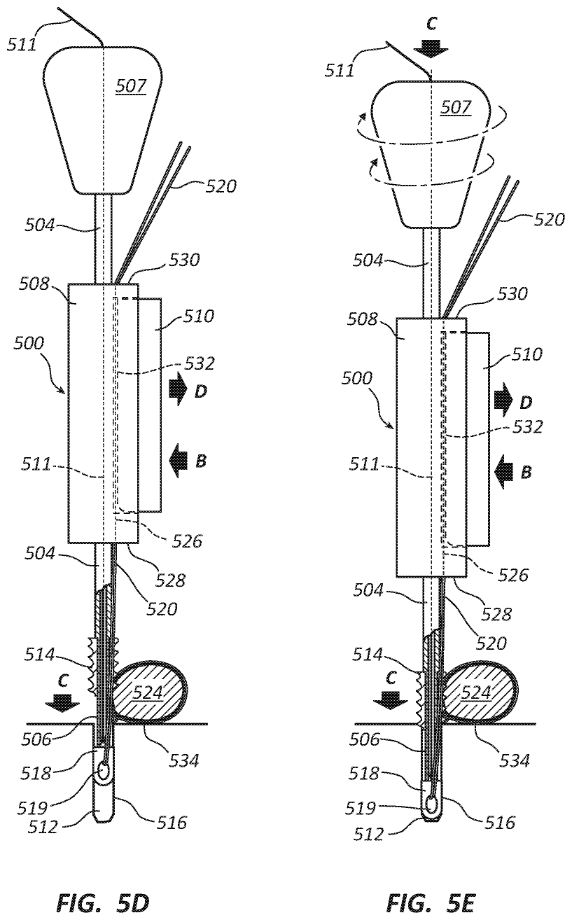

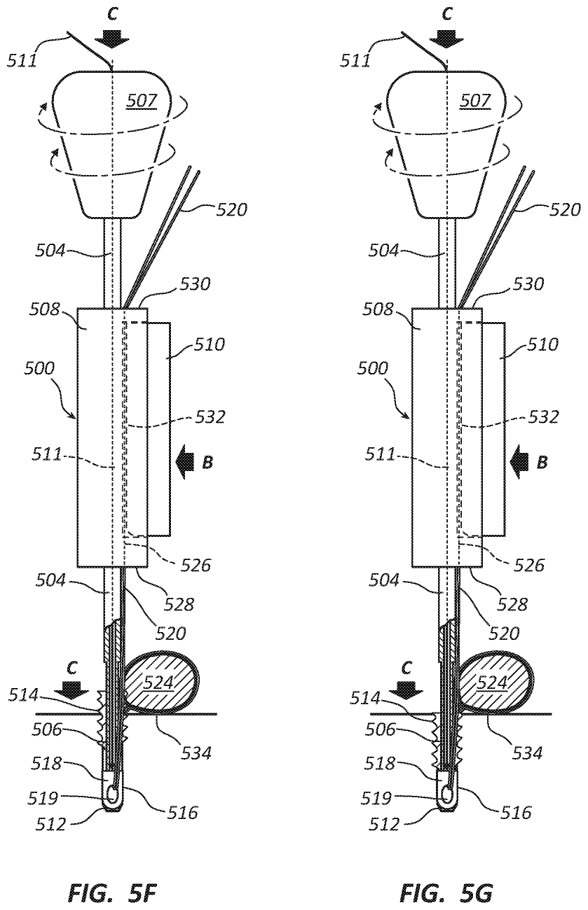

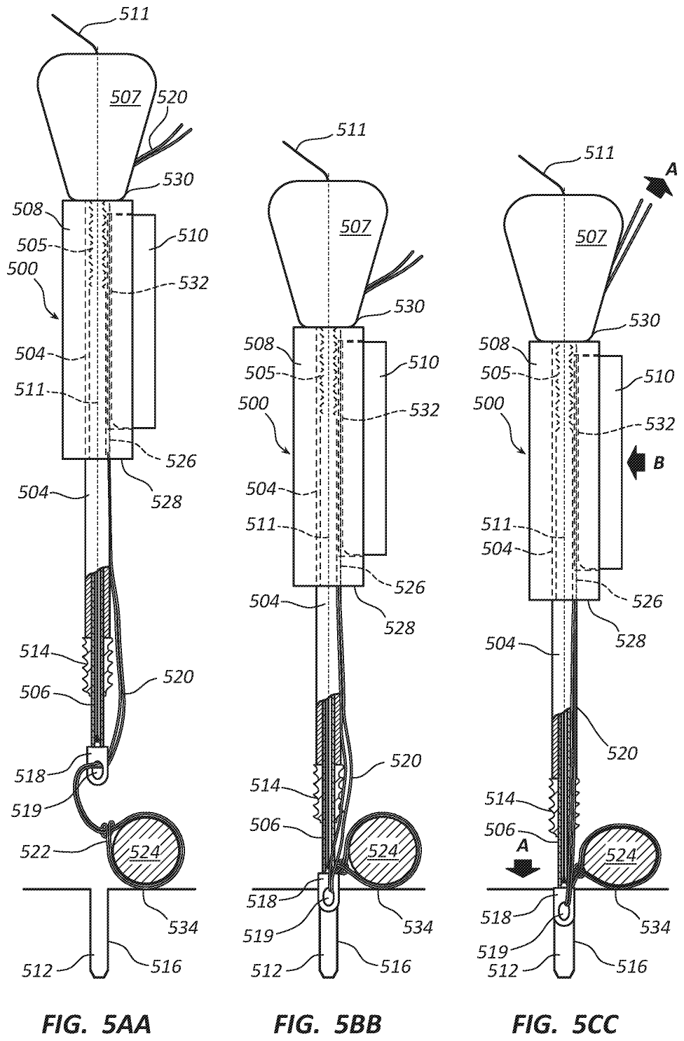

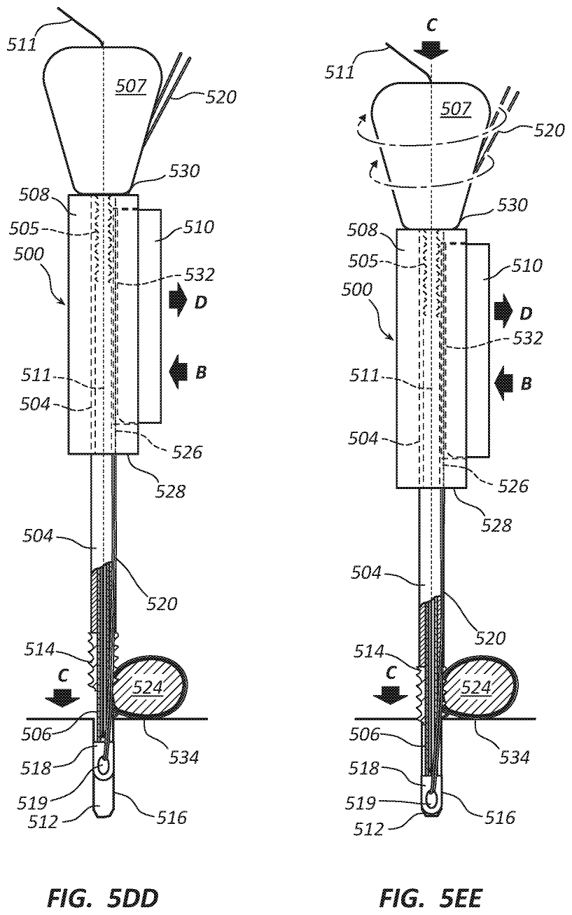

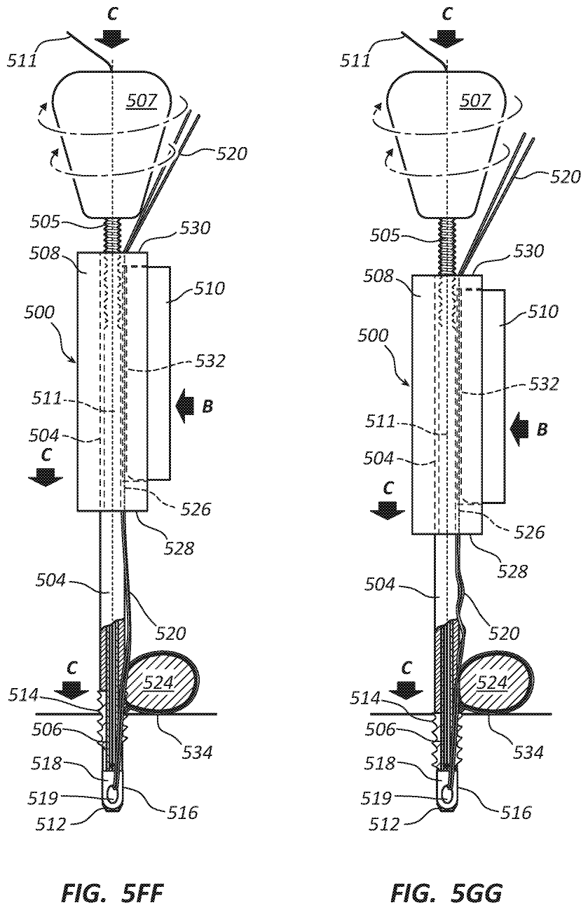

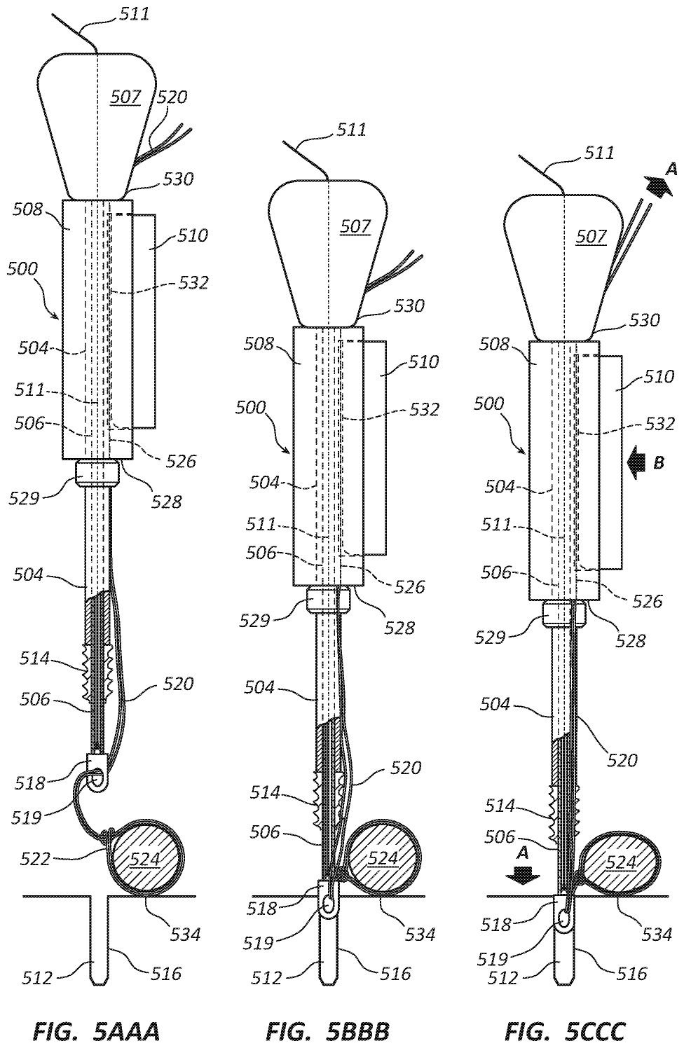

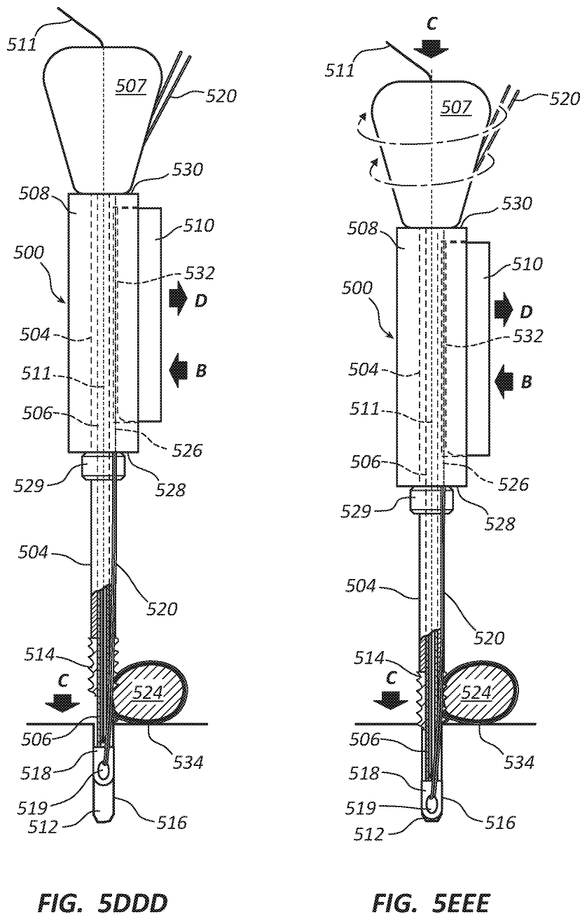

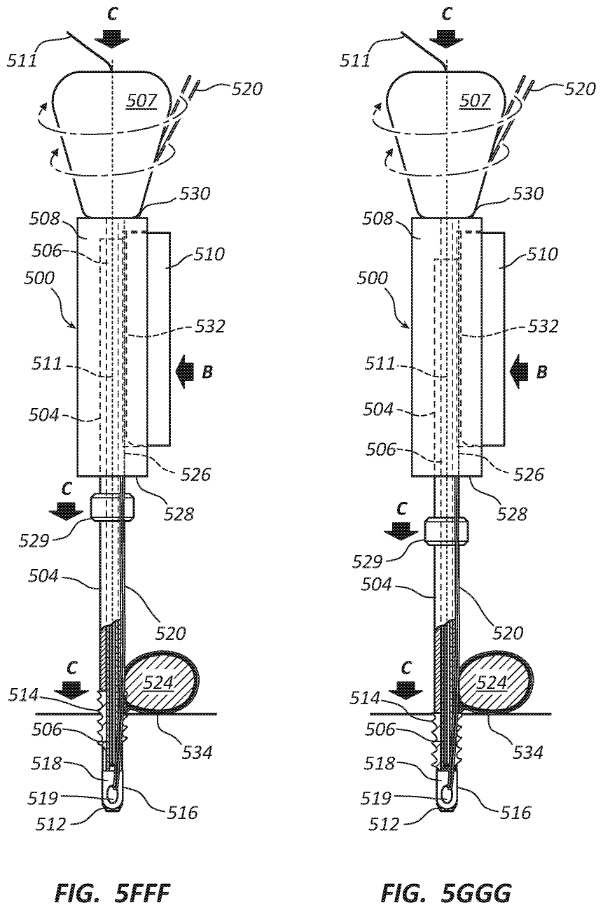

The driver apparatus illustrated in FIGS. 3A-3E can be adapted for use with other suture anchors, such a two-stage push anchors (See FIGS. 4A-4G, 6B-6BB), two-stage threaded anchors (See FIGS. 5A-5G, 5AA-5GG, 5AAA-5GGG, 6C-6CC), or knotless suture anchors having internal locking means (See FIGS. 6D-6DDD). This demonstrates that the inventive driver apparatus is versatile and can be used with a range of different suture anchors while permitting adjustments to suture tension using two hands, e.g., without having to let go of the driver handle and/or mallet to adjust suture tension and/or without assistance from a third hand.

FIGS. 4A-4G illustrate an embodiment of a driver apparatus 400 adapted for use with a two-stage push anchor 402 having an eyelet portion 418 and a separate barbed portion 414 (see FIGS. 6B-6BB for more detail). Driver apparatus 400 includes a second stage drive shaft 404 having a distal end that abuts barbed portion 414 of anchor 402 and a first stage drive shaft 405 slidably received within second stage drive shaft 404 and having a driver tip 406 at the distal end that releasably engages eyelet portion 418 of anchor 402. A driver handle 408 is slidably attached around a portion of second stage drive shaft 404 and attached to first stage drive shaft 405 in a way that prevents relative longitudinal movement of the two by a detent or other attachment means passing through a slot formed in second stage drive shaft 404 (not shown). A manually actuatable suture brake 410 is associated with driver handle 408. Suture brake 410 includes a braking surface 432 and is configured to be selectively actuated and released using the same hand that grips driver handle 408, which frees up the other hand to drive suture two-stage push anchor 402 into a bone tunnel 412.

Similar to single stage push anchor 302 of FIGS. 3A-3E, two stage push anchor 402 includes a barbed portion 414 with barbs configured to engage and obtain purchase with an inner wall 416 of bone tunnel 412 to prevent or resist pullout and an eyelet portion 418 that includes a hole 419 through which suture(s) 422 attached to soft tissue 424 can be threaded. The difference is that eyelet portion 418 is inserted into bone tunnel 412 during a first stage using first stage drive shaft 405, and barbed portion 414 is thereafter driven into bone tunnel 412 during a second stage using second stage drive shaft 405, as explained below.

Eyelet portion 418 can be attached to driver tip 406 using any known attachment means, such as a friction fit, slidable fit, or threaded fit, such as is illustrated in FIGS. 6B-6BB, in which a threaded tip 606′ is received which a threaded socket of LU o eyelet portion 618′. In a friction fit or slidable fit configuration, driver tip 406 can have a non-threaded tip with a cross-sectional shape (e.g., circular, rectangular, pentagonal, hexagonal, or star-shaped) that fits into a correspondingly-shaped socket at a top portion of eyelet portion 418.

Driver handle 408 includes a suture passageway 426 for receiving free suture ends 420. In the illustrated embodiment, passageway 426 extends between a distal end 428 and a proximal end 430 of driver handle 408, although handle 408 can be configured so that passageway 426 opens and/or exits at one or more other locations. Handle 408 may include a side slot (not shown) that permits quick placement of free suture ends 420 within passageway 426. Suture passageway 426 and suture brake 410 are configured such that manual application of pressure to suture brake 410 by the same hand gripping driver handle 408 causes a braking surface 432 to press against and engage free suture ends 420 with sufficient force to substantially prevent or minimize longitudinal movement of free suture ends 420 relative to driver handle 408. Selectively actuating or releasing suture brake 410 permits a surgeon to insert two-stage push anchor 402 into bone tunnel 412 in two stages while selectively increasing, decreasing, or maintaining tension applied by suture(s) 422 to soft tissue 424.

As illustrated in FIG. 4A, sutures 422 are initially threaded through, attached to, and/or wrapped around and then tied to soft tissue 424, which can be a torn labrum. Free suture ends 420 are threaded through hole 419 of eyelet portion 418, which is releasably attached to driver tip 406 of first stage drive shaft 405, and positioned within passageway 426 of driver handle 408. Suture brake 410 may be temporarily removed, retracted, or otherwise positioned to facilitate placement of free suture ends 420 within passageway 426. With handle 408, brake 410, and free suture ends 420 properly assembled, driver apparatus 400 is ready for use in driving two-stage push anchor 402 into bone tunnel 412 while selectively adjusting suture tension.

As illustrated in FIG. 4B, a first step involves placing eyelet portion 418 of two-stage push anchor 402 into the opening of bone tunnel 412. This can be done by gripping handle 408 with one hand and placing eyelet portion 418 at the opening of bone tunnel 412 by means of first stage drive shaft 405. At this stage, free suture ends 420 will typically be loose and have slack. Slack can be taken up by pulling free suture ends 420 using the other hand.

As illustrated in FIG. 4C, a second step involves applying a tensile force A to the portion free suture ends 420 extending from driver handle 408 to take up suture slack and cause free suture ends 420 to become taught. This can be done by gripping driver handle 408 with one hand and pulling on free suture ends 420 with the other hand. Because free suture ends 420 of suture(s) 422 are threaded through eyelet hole 419 of eyelet portion 418, the upward tensile force A applied to free suture ends 420 extending from driver handle 408 is transmitted by eyelet portion 418 to the portion of suture(s) 422 attached to soft tissue 424. This results in downward tensile force A of essentially equal magnitude, which pulls soft tissue 424 toward bone surface 434 adjacent to bone tunnel 412 (assuming minimal friction or other impediment to equalization of tensile force A throughout suture(s) 422).

Depending on the magnitude of tensile force A and the compressibility of soft tissue 424, downward tensile force A may be sufficient to cause deformation and compression of soft tissue 424, resulting in an initial suture tension. In some cases, the initial suture tension may already be the optimal tension. Releasing suture brake 410 when pushing eyelet portion 418 into bone hole 412 permits suture(s) 422 to slide through eyelet hole 419 to increase the length of suture(s) 422 between eyelet portion 418 and soft tissue 424 to maintain or reduce suture tension as eyelet portion 418 is advanced into bone tunnel 412. In other cases, it may be necessary to further increase suture tension. This can be accomplished by pushing eyelet portion 418 into bone tunnel 412 by applying downward force to handle 408, which pushes eyelet portion 418 into bone tunnel 412, while selectively actuating suture brake 410.

As illustrated in FIGS. 4D and 4E, eyelet portion 418 of two-stage push anchor 402 can be pushed into bone tunnel 412 in a first stage by applying a downward force C to handle 408, which is transmitted through first stage drive shaft 405 and driver tip 406 to eyelet portion 418. Downward force C can be applied manually by gripping and pushing handle 408 toward bone tunnel 412 and/or by tapping a spacer 407 attached to the proximal end of second stage drive shaft 404 using a mallet (not shown). Because handle 408 and first stage drive shaft 405 are rigidly attached, the downward force C applied to handle 408 and eyelet portion 418 are essentially the same. By selectively actuating or releasing suture brake 410 (represented by manual squeezing force B and release action D) in order for braking surface 432 to engage or release free suture ends 420, the surgeon can selectively increase, maintain, or reduce suture tension while pushing eyelet portion 418 of two-stage push anchor 402 into bone tunnel 412 using at most two hands: one to grip driver handle 408 and selectively manipulate suture brake 410 and optionally the other to grip a mallet to strike spacer 407. Assistance from a third hand is not required. This provides the surgeon with essentially total control over the acts of inserting eyelet portion 418 of anchor 402 into bone tunnel 412 and adjusting tension on suture(s) 422 and soft tissue 424 without outside assistance.

FIG. 4D illustrates a configuration of a first insertion stage in which eyelet portion 418 has been pushed partially into bone tunnel 412. Barbed portion 414 of two-stage push anchor 402 remains outside of bone tunnel 412 until a second insertion stage, which is illustrated in FIGS. 4F and 4G. As eyelet portion 418 is incrementally pushed into bone tunnel 412, the distance between eyelet portion 418 and soft tissue 424 is also incrementally increased. If suture brake 410 is actuated during eyelet insertion 412, the tension between sutures(s) 422 and soft tissue 424 is increased. Releasing suture brake 410 can maintain or reduce suture tension depending on the timing of such release relative to eyelet 418 being pushed deeper into bone tunnel 412.

FIG. 4E illustrates a final configuration of the first insertion stage in which eyelet portion 418 has been pushed all the way into bone tunnel 412. In this configuration, the maximum distance between eyelet portion 418 and soft tissue 424 is attained and essentially maximum suture tension has been applied to suture(s) 422 and soft tissue 424. However, until barbed portion 414 has been driven into bone tunnel 412 during the second insertion stage, suture tension is not yet fixed and soft tissue 424 is not held against bone surface 434 with a fixed final force. That allows the surgeon to make further adjustments to suture tension if necessary.

If suture tension is determined to be optimal after the first insertion stage, the surgeon can proceed with the second insertion stage to lock suture(s) 422 in place by driving barbed portion 414 into bone tunnel 412, as discussed below relative to FIGS. 4F and 4G, in order to set final suture tension and pull soft tissue 424 against bone surface 434 with final force. Maintaining suture brake 410 in the actuated position maintains the optimal suture tension while driving barbed portion 412 into bone tunnel 412.

If suture tension is determined to be too low, additional suture tension can be applied, such as by releasing suture brake 410, pulling on free suture ends 420 with greater tensile force A, re-actuating suture brake 410 to hold free suture ends 420 with the increased suture tension, and performing the second insertion stage to lock suture(s) 422 against bone tunnel wall 416 by barbed portion 414. Alternatively, the surgeon can partially or fully withdraw eyelet portion 418 from bone tunnel 412 and repeat some or all of the steps of the first insertion stage.

If suture tension is determined to be too high, excessive tension can be reduced by releasing suture brake 410 while pulling on free suture ends 420 with less tensile force A to apply a desired reduced tension and prevent excessive loosening, re-actuating suture brake 410 to hold free suture ends 420 with the reduced suture tension, and performing the second insertion stage to lock suture(s) 422 against bone tunnel wall 416 by barbed portion 414.

FIGS. 4F and 4G illustrate barbed portion 414 of two-stage push anchor 402 being driven into bone tunnel 412 in the second insertion stage using second stage drive shaft 404 in order for barbs to engage inner wall 416 of bone tunnel 412 and lock sutures 422 in place with fixed tension and pull soft tissue 424 against bone surface 434 with fixed force.

FIG. 4F illustrates a configuration of the second insertion stage in which barbed portion 414 of two-stage push anchor 402 has been driven partially into bone tunnel 412. With spacer 407 removed and while gripping handle 408 and actuating suture brake 410 with one hand, the surgeon can drive barbed portion 414 into bone tunnel 412 by striking a proximal head end 409 of second stage drive shaft 404 with a mallet. During the second insertion stage, first stage drive shaft 405 and eyelet portion 418 are maintained at the same longitudinal relationship relative to handle 408 which, together with application of a braking force by actuating suture brake 410, maintains essentially constant suture tension as barbed portion 414 is driven into bone tunnel 412. Part of barbed portion 414 of two-stage push anchor 402 is shown engaging inner wall 416 of bone tunnel 412. Each tap of the mallet drives barbed portion 414 progressively deeper into bone tunnel 412, causing more of barbed portion 414 to engage and obtain purchase with bone tunnel wall 416.

FIG. 4G illustrates a final configuration of the second insertion stage in which barbed portion 414 has been driven all the way into bone tunnel 412 using several additional taps of the mallet. In the final configuration, all of barbed portion 414 of two-stage push anchor 402 engages inner bone tunnel wall 416, gaining maximum purchase to prevent or limit pullout of anchor 402 from bone tunnel 412. This locks suture(s) 422 within bone tunnel 412 at a final suture tension and holds soft tissue 424 against bone surface 434 with a final force.

In summary, FIGS. 4A-4G illustrate how driver apparatus 400 facilitates fine, real time adjustments to suture tension while driving a two-stage push anchor 402 into a bone tunnel 412. By selectively increasing or maintaining suture tension by selective actuating or releasing suture brake 410 while pushing eyelet portion 418 into bone tunnel 412, the surgeon can adjust suture tension and achieve optimal suture tension using at most two hands. As discussed above, the surgeon has several opportunities to adjust and fine tune suture tension and readily achieve optimal suture tension without assistance from a third hand.

FIGS. 5A-5G illustrate an embodiment of a driver apparatus 500 adapted for use with a two-stage threaded anchor 502 having an eyelet portion 518 and a separate threaded portion 514 (see FIGS. 6C-6CC for more detail). Driver apparatus 500 includes a drive shaft 504, a driver tip 506 for engaging eyelet portion 518 and threaded portion 514 of two-stage threaded anchor 502, a driver handle 508 rotatably connected to drive shaft 504 in a fixed longitudinal position along drive shaft 504, a driver knob 507 fixedly attached to drive shaft 504, and a manually actuatable suture brake 510 associated with driver handle 508. Suture brake 510 includes a braking surface 532 and is configured to be selectively actuated and released using the same hand that grips driver handle 508, which frees up the other hand to drive two-stage threaded suture anchor 502 into a bone tunnel 512.

Similar to two-stage stage push anchor 402 of FIGS. 4A-4G, two stage threaded anchor 502 includes an eyelet portion 518 that includes a hole 519 through which suture(s) 522 attached to soft tissue 524 can be threaded, which is inserted into a bone runnel 512 in a first insertion stage. Similar to barbed portion 414 of two-stage stage push anchor 402, threaded portion 514 is driven into bone tunnel 512 in a second insertion stage to lock suture(s) 522 within a bone tunnel 512 fixed tension. However, rather than being pushed into bone tunnel 512, threaded portion 514 is screwed into bone tunnel 512 by rotating drive shaft 504 and driver tip 506 using driver knob 507.

Driver tip 506 has a cross-sectional shape for engaging and turning a correspondingly shaped passageway through threaded portion 514 and a distal opening at the distal end that rotatably and releasably engages a nipple of eyelet portion 518. The cross-sectional shape of driver tip 506 can be any suitable shape, such as rectangular, pentagonal, hexagonal, or star-shaped, and the internal passageway through threaded portion 514 will be correspondingly shaped (e.g., rectangular, pentagonal, hexagonal, or star-shaped) so that rotation of drive shaft 504 and driver tip 506 by rotating driver knob 507 relative to handle 508 causes threaded portion 514 to be rotated and screwed into bone tunnel 512 during a section insertion stage.

A retention thread 511 is attached to a hole in the nipple of eyelet portion 518, passes through an interior lumen of drive shaft 504, and emerges from a proximal end of a driver knob 507. Tension applied to retention thread 511 retains the nipple of eyelet portion 518 within the distal opening of driver tip 506 in a rotatable relationship with driver tip 506. This enables eyelet portion 518 to remain stationary as threaded portion 514 is rotated and screwed into bone tunnel 512. Threaded portion 514 is slidably attached to driver tip 506 longitudinally and retained between eyelet portion 518 and a shoulder of drive shaft 504 where driver tip 506 begins. The slidable connection between threaded portion 514 and driver tip 506 permits threaded portion 514 to slide longitudinally down driver tip 506 as it is screwed and pulled into bone tunnel 512 by turning drive shaft 504 and driver tip 506.

Driver handle 508 includes a suture passageway 526 for receiving free suture ends 520. In the illustrated embodiment, passageway 526 extends between a distal end 528 and a proximal end 530 of driver handle 508, although handle 508 can be configured so that passageway 526 opens and/or exits at one or more other locations. Handle 508 may include a side slot (not shown) that permits quick placement of free suture ends 520 within passageway 526. Suture passageway 526 and suture brake 510 are configured such that manual application of pressure to suture brake 510 by the same hand gripping driver handle 508 causes a braking surface 532 to press against and engage free suture ends 520 with sufficient force to substantially prevent or minimize longitudinal movement of free suture ends 520 relative to driver handle 508. Selectively actuating or releasing suture brake 510 permits a surgeon to insert two-stage screw anchor 502 into bone tunnel 512 in two stages while selectively increasing, maintaining, or decreasing tension applied by suture(s) 522 to soft tissue 524.

As illustrated in FIG. 5A, sutures 522 are initially threaded through, attached to, and/or wrapped around and then tied to soft tissue 524, which can be a torn labrum. Free suture ends 520 are threaded through hole 519 of eyelet portion 518, which is rotatably and releasably attached to the distal end of driver tip 506 by the nipple and retention thread 511, and positioned within passageway 526 of driver handle 508. Suture brake 510 may be temporarily removed, retracted, or otherwise positioned to facilitate placement of free suture ends 520 within passageway 526. With handle 508, brake 510, and free suture ends 520 properly assembled, driver apparatus 500 is ready for use in driving two-stage threaded anchor 502 into bone tunnel 512 while selectively adjusting suture tension.

As illustrated in FIG. 5B, a first step involves placing eyelet portion 518 of two-stage threaded anchor 502 into the opening of bone tunnel 512. This can be done by gripping handle 508 with one hand and placing eyelet portion 518 at the opening of bone tunnel 512 by means of driver tip 506. At this stage, free suture ends 520 will typically be loose and have slack. Slack can be taken up by pulling free suture ends 520 using the other hand.

As illustrated in FIG. 5C, a second step involves applying a tensile force A to the portion free suture ends 520 extending from driver handle 508 to take up suture slack and cause free suture ends 520 to become taught. This can be done by gripping driver handle 508 with one hand and pulling on free suture ends 520 with the other hand. Because free suture ends 520 of suture(s) 522 are threaded through eyelet hole 519 of eyelet portion 518, the upward tensile force A applied to free suture ends 520 extending from driver handle 508 is transmitted by eyelet portion 518 to the portion of suture(s) 522 attached to soft tissue 524. This results in downward tensile force A of essentially equal magnitude, which pulls soft tissue 424 toward bone surface 534 adjacent to bone tunnel 512 (assuming minimal friction or other impediment to equalization of tensile force A throughout suture 522).

Depending on the magnitude of tensile force A and the compressibility of soft tissue 524, downward tensile force A may be sufficient to cause deformation and compression of soft tissue 524, resulting in an initial suture tension. In some cases, the initial suture tension may already be the optimal tension. Releasing suture brake 510 when pushing eyelet portion 518 into bone hole 512 permits suture(s) 522 to slide through eyelet hole 519 to increase the length of suture(s) 522 between eyelet portion 518 and soft tissue 524 to maintain or reduce suture tension as eyelet portion 518 is advanced into bone tunnel 512. In other cases, it may be necessary to further increase suture tension. This can be accomplished by pushing eyelet portion 518 into bone tunnel 512 by applying downward force to handle 508 and/or drive knob 507, which pushes eyelet portion 518 into bone tunnel 512, while selectively actuating suture brake 510.

As illustrated in FIGS. 5D and 5E, eyelet portion 518 of two-stage threaded anchor 502 can be pushed into bone tunnel 512 in a first stage by applying a downward force C to handle 508 and/or drive knob 507, which is transmitted through drive shaft 504 and driver tip 506 to eyelet portion 518. Downward force C can be applied manually by gripping and pushing handle 508 and/or driver knob 507 toward bone tunnel 512 and/or by tapping drive knob 507 attached to the proximal end of drive shaft 504 using a mallet (not shown). Because driver knob 507 and drive shaft 506 are rigidly attached, the downward forces C applied to driver knob 507 and/or handle 508 and eyelet portion 518 are essentially the same. By selectively actuating or releasing suture brake 510 (represented by manual squeezing force B and release action D) in order for braking surface 532 to engage or release free suture ends 520, the surgeon can selectively increase, maintain, or reduce suture tension while pushing eyelet portion 518 of two-stage threaded anchor 502 into bone tunnel 512 using at most two hands: one to grip driver handle 508 and selectively manipulate suture brake 510 and optionally the other to grip driver knob 507 and/or a mallet used to strike driver knob 507. Assistance from a third hand is not required. This allows the surgeon to have essentially total control over the acts of inserting eyelet portion 518 of anchor 502 into bone tunnel 512 and adjusting tension on suture(s) 522 and soft tissue 524 without outside assistance.

FIG. 5D illustrates a configuration of a first insertion stage in which eyelet portion 518 has been pushed partially into bone tunnel 512. Threaded portion 514 of two-stage threaded anchor 502 remains outside of bone tunnel 512 until a second insertion stage, which is illustrated in FIGS. 5F and 5G. As eyelet portion 518 is incrementally pushed into bone tunnel 512, the distance between eyelet portion 518 and soft tissue 524 is also incrementally increased. If suture brake 510 is actuated during eyelet insertion 512, the tension between sutures(s) 522 and soft tissue 524 is increased. Releasing suture brake 510 can maintain or reduce suture tension depending on the timing of such release relative to eyelet 518 being pushed deeper into bone tunnel 512.

FIG. 5E illustrates a final configuration of the first insertion stage in which eyelet portion 518 has been pushed all the way into bone tunnel 512. In this configuration, the maximum distance between eyelet portion 518 and soft tissue 524 is attained and essentially maximum suture tension has been applied to suture(s) 522 and soft tissue 524. However, until threaded portion 514 has been screwed into bone tunnel 512 during the second insertion stage, suture tension is not yet fixed and soft tissue 524 is not held against bone surface 534 with a fixed final force. That allows the surgeon to make further adjustments to suture tension if necessary.

If suture tension is determined to be optimal after the first insertion stage, the surgeon can proceed with the second insertion stage to lock free suture ends 520 in place by screwing threaded portion 514 into bone tunnel 512, as discussed below relative to FIGS. 5F and 5G, in order to set final suture tension and pull soft tissue 524 against bone surface 534 with final force. Maintaining suture brake 510 in the actuated position maintains the optimal suture tension while screwing threaded portion 512 into bone tunnel 512.

If suture tension is determined to be too low, additional suture tension can be applied, such as by releasing suture brake 510, pulling on free suture ends 520 with greater tensile force A, re-actuating suture brake 510 to hold free suture ends 520 with the increased suture tension, and performing the second insertion stage to lock suture(s) 522 against bone tunnel wall 516 by threaded portion 514. Alternatively, the surgeon can partially or fully withdraw eyelet portion 518 from bone tunnel 512 and repeat some or all of the steps of the first insertion stage.

If suture tension is determined to be too high, excessive tension can be reduced by releasing suture brake 510 while pulling on free suture ends 520 with less tensile force A to apply a desired reduced tension and prevent excessive loosening, re-actuating suture brake 510 to hold free suture ends 520 with the reduced suture tension, and performing the second insertion stage to lock suture(s) 522 against bone tunnel wall 516 by threaded portion 514.

FIGS. 5F and 5G illustrate threaded portion 514 of two-stage threaded anchor 502 being driven (i.e., screwed) into bone tunnel 512 in the second insertion stage using driver tip 506 in order for threads to engage inner wall 516 of bone tunnel 512 and lock sutures 522 in place with fixed tension and pull soft tissue 524 against bone surface 534 with fixed force.

FIG. 5F illustrates a configuration of the second insertion stage in which threaded portion 514 of two-stage threaded anchor 502 has been driven (i.e., screwed) partially into bone tunnel 512. While gripping handle 508 and actuating suture brake 510 with one hand and gripping driver knob 507 with the other, the surgeon can drive threaded portion 514 into bone tunnel 512 by turning driver knob 507 to rotate drive shaft 504 and driver tip 506 relative to handle 508 and screw threaded portion 514 into bone tunnel 512. Initially, the shoulder of drive shaft 504 where driver tip 506 extends into threaded portion 514 abuts the proximal end of threaded portion 514 and urges it into bone tunnel 512 to obtain purchase with interior bone tunnel wall 516. Once threaded portion 514 has obtained sufficient purchase with bone tunnel wall 516, threaded portion 514 will screw into bone tunnel 512 on its own and slide longitudinally down driver tip 506 as it is turned by rotating driver knob 507. During the second insertion stage, drive shaft 504, driver tip 506, and eyelet portion 518 are maintained at the same longitudinal relationship relative to handle 508 which, together with application of braking force by actuating suture brake 510, maintains essentially constant suture tension as threaded portion 514 is screwed into bone tunnel 512. Part of threaded portion 514 of two-stage threaded anchor 502 is shown engaging inner wall 516 of bone tunnel 512. Each rotation of driver knob 507 drives threaded portion 514 progressively deeper into bone tunnel 512, causing more of threaded portion 514 to engage and obtain purchase with bone tunnel wall 516.

FIG. 5G illustrates a final configuration of the second insertion stage in which threaded portion 514 has been driven all the way into bone tunnel 512 by additional rotations of driver knob 507. In the final configuration, all of threaded portion 514 of two-stage threaded anchor 502 engages inner bone tunnel wall 516, gaining maximum purchase to prevent or limit pullout of anchor 502 from bone tunnel 512. This locks suture(s) 522 within bone tunnel 512 at a final suture tension and holds soft tissue 524 against bone surface 534 with a final force.

In summary, FIGS. 5A-5G illustrate how driver apparatus 500 facilitates fine, real time adjustments to suture tension while driving a two-stage threaded anchor 502 into a bone tunnel 512. By selectively increasing or maintaining suture tension by selective actuating or releasing suture brake 510 while pushing eyelet portion 518 into bone tunnel 512, the surgeon can adjust suture tension and achieve optimal suture tension using at most two hands. As discussed above, the surgeon has several opportunities to adjust and fine tune suture tension and readily achieve optimal suture tension without assistance from a third hand.

FIGS. 5AA-5GG illustrate a modified embodiment of driver apparatus 500 that is analogous to driver 400 in that it includes a first stage drive shaft 505, 506 for pushing eyelet 518 into bone tunnel 512 in a first insertion stage and a second stage drive shaft 504 that maintains abutment with a threaded portion 514 of two-stage threaded anchor 502 in a second insertion stage in order to assist driving threaded portion 514 into bone tunnel 512. First stage drive shaft 505, 506 is fixed to driver knob 507 and slidable and rotatably received within a passageway through second stage drive shaft 504. Second stage drive shaft 504 fixed to handle 508.

The first stage drive shaft extends between proximal and distal ends and includes a proximal threaded shaft 505 fixedly attached to driver knob 507 and a distal driver tip 506 fixedly attached to threaded shaft 505 so as to remain at a fixed longitudinal distance relative to driver knob 507 and such that proximal threaded shaft 505 and distal driver tip 506 rotate together with driver knob 507. Proximal threaded shaft 505 engages a correspondingly threaded passageway through a proximal interior portion of handle 508, with threads having the same pitch as the threads in threaded portion 514 of two-stage threaded anchor 502, but with an opposite angle so that proximal threaded shaft 505 is withdrawn from handle 508 when driver knob 507 is rotated clockwise relative to handle 508. This, in turn, causes handle 508 and second stage drive shaft 504 to move away from handle portion 507 at the same rate at which threaded portion 514 is screwed into bone tunnel 512. This maintains a constant distance between handle 508 and threaded portion 514 and maintains abutment between a distal end surface of second stage drive shaft 504 and a proximal surface of threaded portion 514. In this way, second stage drive shaft 504 is able to abut and maintain pressure against threaded portion 514 while being screwed into bone tunnel 512.

An advantage of the embodiment of driver apparatus 500 in FIGS. 5AA-5GG compared to the embodiment of driver apparatus 500 in FIGS. 5A-5G is that second stage drive shaft 504 pushes against and assists in screwing threaded portion 514 into bone tunnel 512. This prevents inadvertent stripping of bone tunnel wall 516 and/or pullout of threaded portion 514 from bone tunnel 512 during the second insertion stage. A disadvantage is that handle 508 moves toward eyelet portion 518 as it is advanced in unison with threaded portion 514 when threaded portion is screwed into bone tunnel 512. This reduces the distance between handle 508 and eyelet portion 518, which releases the tension on free suture ends 520 between handle 508 and threaded portion 514. As a result, constant suture tension cannot be maintained by actuating brake 510 when threaded portion 514 is advanced into bone tunnel 512. It is believed, however, that this is not a significant problem. Once threaded portion 514 has obtained sufficient purchase with bone tunnel wall 516 to threadably engage and create corresponding negative threads in bone tunnel wall 516, the same force that compresses against bone tunnel wall 516 to create the corresponding negative threads will frictionally engage suture(s) 522 and maintain essentially constant tension between eyelet portion 518 and soft tissue 524.

FIGS. 5AA-5GG show similar stages of insertion of two-stage threaded anchor 502 using modified driver apparatus 500 as those shown in FIGS. 5A-5G. FIG. 5AA shows the assembled driver-anchor assembly ready for use. It can be assembled in similar fashion as described above relative to FIG. 5A. FIGS. 5BB-5EE illustrate the first insertion stage in which eyelet portion 518 is inserted and advanced into bone tunnel 512, while the surgeon applies an initial suture tension in FIG. 5CC and selectively actuates and releases brake 510 in FIGS. 5DD and 5EE to set a desired suture tension and pull soft tissue 524 toward bone surface 534. FIGS. 5FF and 5GG show threaded portion 514 being screwed into bone tunnel 512 and advanced relative to bone tunnel wall 516.

The main difference in the acts shown in FIGS. 5AA-5GG relative to those shown in FIGS. 5A-5G is that in FIGS. 5FF and 5GG, second stage drive shaft 504 remains in abutment with threaded portion 514 the second insertion stage to assist driving threaded portion 514 into bone tunnel 512. This occurs as a result of handle 508 moving away from driver knob 507 at the same rate as threaded portion 514 is advanced into bone tunnel 512 as driver knob 507 is rotated relative to handle 508. This also means handle 508 is advanced toward eyelet portion 518 at the same or similar rate as threaded portion 514 is screwed into bone tunnel 512 and advanced toward eyelet portion 518. This causes free suture ends 520 between handle 508 and threaded portion 514 to relax and release tension. However, the tension of suture(s) 522 between eyelet portion 518 and soft tissue 524 remains substantially constant as a result of the frictional forces exerted by threaded section 514, which press and frictionally lock suture(s) 522 against bone tunnel wall 516. In other words, the same forces exerted by threaded portion 514 to compress bone tunnel wall 516 and create the corresponding negative threads can frictionally engage suture(s) 522 and maintain substantially constant suture tension between eyelet portion 518 and soft tissue 524, thus substantially maintaining the suture tension that is set by the surgeon when performing the steps illustrated in FIGS. 5CC-5EE.

FIGS. 5AAA-5GGG illustrates a third embodiment of a driver apparatus 500 that provides the benefits of maintaining suture tension when screwing in threaded section 514 as shown in FIGS. 5A-5G while maintaining abutment between second stage drive shaft 504 and threaded portion 514 as shown in FIGS. 5AA-5GG. Driver apparatus 500 in this embodiment includes a driver knob 507 rotatably but non-threadably connected to handle 508. This maintains driver knob 507 and handle 508 in a constant longitudinal relationship as driver knob 507 is rotated in order to screw threaded anchor portion 514 into bone tunnel 512. A second stage drive shaft 504 is configured to move away from handle 508 and advance together with threaded anchor portion 514 as threaded portion 514 is advanced into bone tunnel 512. Thus, drive shaft 504 abuts and exerts pressure against threaded portion 514 to assist in screwing it into bone tunnel regardless of whether sufficient purchase has been achieve with bone tunnel wall 516 to cause threaded portion 514 to threadably advance into bone tunnel.

Driver apparatus 500 of this embodiment includes a first stage drive shaft 506 for pushing eyelet 518 into bone tunnel 512 during the first insertion stage and the aforementioned second stage drive shaft 504 that maintains abutment with threaded anchor portion 514 during the second insertion stage assist the driving of threaded portion 514 into bone tunnel 512. First stage drive shaft 506 is fixed to driver knob 507 and slidable and rotatably received within a passageway through second stage drive shaft 504.

Second stage drive shaft 504 includes a proximal threaded portion (not visible) that engages a correspondingly threaded passageway through a distal interior portion of handle 508. The threads on the proximal threaded portion of second stage drive shaft 504 and the corresponding threads within the distal interior portion of handle 508 can have the same pitch and angle as the threads on threaded anchor portion 514. This causes second stage drive shaft 504 to move away from handle 508 and advance in unison with threaded anchor portion 514 as threaded portion 514 is advanced into bone tunnel 512. Because handle 508 does not move away from driver knob 507, actuating brake 510 maintains constant tension on free suture ends 520 even as threaded anchor portion 514 and second stage drive shaft 504 move in unison away from handle 508 as driver knob 507 is rotated (e.g., clockwise) by the surgeon to drive and advance threaded portion 514 into bone tunnel 512.

FIGS. 5AAA-5GGG show similar stages of insertion of two-stage threaded anchor 502 using third modified driver apparatus 500 as those shown in FIGS. 5A-5G and in FIGS. 5AA-5GG. FIG. 5AAA shows the assembled driver-anchor assembly ready for use. It can be assembled in similar fashion as described above relative to FIG. 5A. FIGS. 5BBB-5EEE illustrate the first insertion stage in which eyelet portion 518 is inserted and advanced into bone tunnel 512, while the surgeon applies an initial suture tension in FIG. 5CCC and selectively actuates and releases brake 510 in FIGS. 5DDD and 5EEE to set a desired suture tension and pull soft tissue 524 toward bone surface 534. FIGS. 5FFF and 5GGG show threaded portion 514 being screwed into bone tunnel 512 and advanced relative to bone tunnel wall 516. Because handle 508 stays the same distance from eyelet portion 518 when threaded portion 514 is advance into bone tunnel 512, suture tension is maintained as long as brake 510 is actuated. Because second stage drive shaft 504 moves longitudinally together with threaded portion 514 as it is advanced into bone tunnel 512, second stage drive shaft 504 pushes against and assists in driving threaded portion 514 into bone tunnel.

FIGS. 6A-6BB illustrate exemplary embodiments of knotless push anchors that can be used with inventive driver apparatus disclosed herein. FIGS. 6A-6AA illustrate a single stage knotless push anchor 602, and FIGS. 6B-6BB illustrate a two-stage knotless push anchor 602′. Suitable driver apparatus, such as driver apparatus 300 and driver apparatus 400, are configured for use with said anchors, respectively. Although driver tips 606 and 606′ are illustrated in 6A-6BB as being threaded, they can be non-threaded so as to frictionally and/or slidably engage corresponding recesses in eyelet 618 and eyelet portion 618′.

The single stage knotless push anchor 602 illustrated in FIGS. 6A and 6AA includes a barbed portion 614 with a plurality of barbs that enable anchor 602 to engage and obtain purchase with bone tissue in a bone tunnel wall as anchor 602 is driven into a bone tunnel in order to prevent the anchor from disengaging and pulling out from the bone tunnel. Barbed portion 614 also fixes suture tension. Anchor 602 is a single stage anchor because it is inserted into the bone tunnel as a single piece, as discussed above relative to FIGS. 3A-3E.

The two-stage push anchor 602′ illustrated in FIGS. 6B-6BB includes an eyelet portion 618′ with an eyelet hole 619′ therethrough for receiving suture(s) 622. When using two-stage bone anchor 602′, free suture ends 620 of suture(s) 622 attached to soft tissue (not shown) are threaded through eyelet hole 619′. Eyelet portion 618′ of anchor 602′ can then be pushed into a bone tunnel in a first insertion stage (e.g., bone tunnel 412 in FIGS. 4A-4G). Thereafter, barbed portion 614′ can be driven into the bone tunnel next to eyelet portion 618′ to lock bone anchor 602′ and suture(s) 622 in place within the bone tunnel and fix suture tension being exerted on the soft tissue.

FIGS. 6C-6CC illustrate an embodiment of a two-stage threaded suture anchor 602″ used with a driver apparatus 500 as illustrated in FIGS. 5A-5G. Two-stage threaded suture anchor 602″ can maintain the same configuration even if used with the modified driver apparatus 500 illustrated in FIGS. 5AA-5GG or the modified driver apparatus 500 illustrated in FIGS. 5AAA-5GGG. Two-stage threaded suture anchor 602″ comprises an eyelet portion 618″, similar to eyelet portion 618′ of two-stage push anchor 602′. Eyelet portion 618″ includes an eyelet hole 619″ through which free suture ends 620 of suture(s) 622 attached to soft tissue (not shown) are threaded. After free suture ends of suture 620 are threaded through eyelet hole 619″, eyelet portion 618″ of anchor 602″ can be pushed into the bone tunnel in a first insertion stage (e.g., into bone tunnel 512 in FIGS. 5A-5G, 5AA-5GG, and 5AAA-5GGG). Then, threaded portion 614″ is driven (i.e., screwed) into bone tunnel 612 and toward eyelet portion 618″ to lock the bone anchor 602″ and suture(s) 622 in place within the bone tunnel and fix suture tension being exerted on the soft tissue.

FIGS. 6D-6DDD illustrate an embodiment of an internal locking knotless suture anchor 660 that can be used with driver apparatus having a manually actuatable suture brake. An example is the Cinchlock Knotless Labrum Anchor of Stryker. Rather than locking free suture ends 620 in place between barbs or threads and the bone tunnel wall, such as with anchors 602, 602′ and 602″, suture anchor 660 includes an interior locking 676 which slides within a passageway of internal knotless locking suture anchor 660 in order to “lock” the suture 620 within the interior of knotless suture anchor 660. While a driver sleeve 666 pushes the anchor 660 into the bone tunnel, the interior locking portion 676 is pulled towards the proximal end of the anchor 660 and engages with a cleat that locks suture 620 within the interior of suture anchor 660.

FIGS. 7A-7F illustrate an example embodiment of a driver apparatus 700 and an example method of using of driver 700 to drive a single stage push anchor 702 into a bone tunnel 712, such as the push anchor illustrated in FIGS. 3A-3D and 6A-6AA. Driver 700 includes a driver handle 708 and a manually actuatable suture brake 710 associated with driver handle 708 for adjusting suture tension while driving anchor 702 into bone tunnel 712.

Driver apparatus 700 includes an elongated drive shaft 704 rigidly affixed to driver handle 708. The handle 708 is configured to receive suture brake 710, which includes a braking surface 732 that can selectively apply pressure to and engage free suture ends 720 positioned in a suture passageway 726 within handle 708. Drive shaft 704 includes a driver tip at the distal end for releasably engaging push anchor 702 during insertion. In some embodiments, the driver tip may be an interchangeable piece which can be changed to accommodate various suture anchor designs.

Suture brake 710 can be mechanically associated with driver handle 708, such as by being received within an elongated slot 750. In some embodiments, suture 710 is free to slide in and out within slot 750 without a spring or spring-like mechanism. The driver handle 708 further comprises an interior wall 752 at the base of slot 750, which provides a surface against which braking surface 732 can be pressed in order to prevent or minimize longitudinal movement of free suture ends 720 of a suture 722 relative to handle 708.

The free ends 720 of suture 722 can be initially threaded an eyelet hole 719 in eyelet portion 718 of push anchor 702 and then threaded through a suture passageway 726 that opens through a distal side 728 of handle 708 and into slot 750. The free suture ends 620 are positioned between braking surface 732 of suture brake 710 and interior wall 752 can and exit through an opening in proximal side 730 of handle 708. With the surgeon gripping handle 708 with a first hand, a second hand can pull free suture ends 620 to apply initial suture tension. The surgeon can manually squeeze handle 708 to actuate suture brake 710 in order for braking surface 732 to engage with and restrain longitudinal movement of free suture ends 720 relative to handle 708. Gripping handle 708 and selectively actuating brake 710 with one hand, the user's other hand is available to perform other procedural tasks, such as using a mallet to tap on proximal end 730 of handle 708 to drive bone anchor 702 into bone tunnel 712.

As push anchor 702 is driven into bone tunnel 712, the surgeon can selectively actuate and release suture brake 710 selectively increase or maintain suture tension. This permits real time adjustments to suture tension and correct positioning of the damaged connective tissue in order to promote healing, while securing push anchor 702 within bone tunnel 702. Once anchor 702 has been driven into bone tunnel 712, a blade or other cutting device 760 can be used to trim free suture ends emerging from the patient's skin.

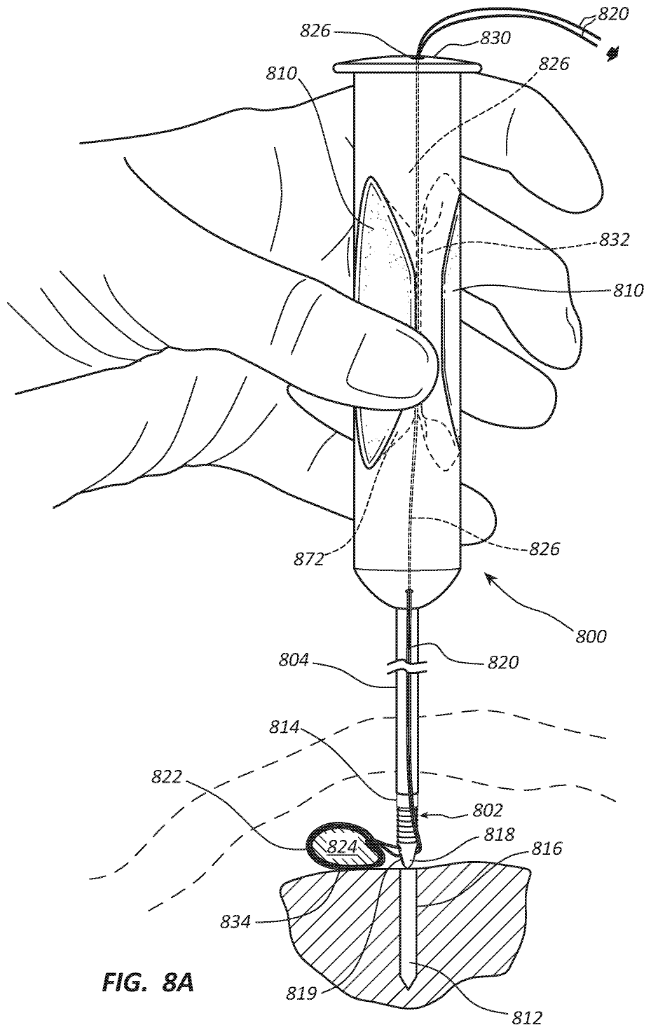

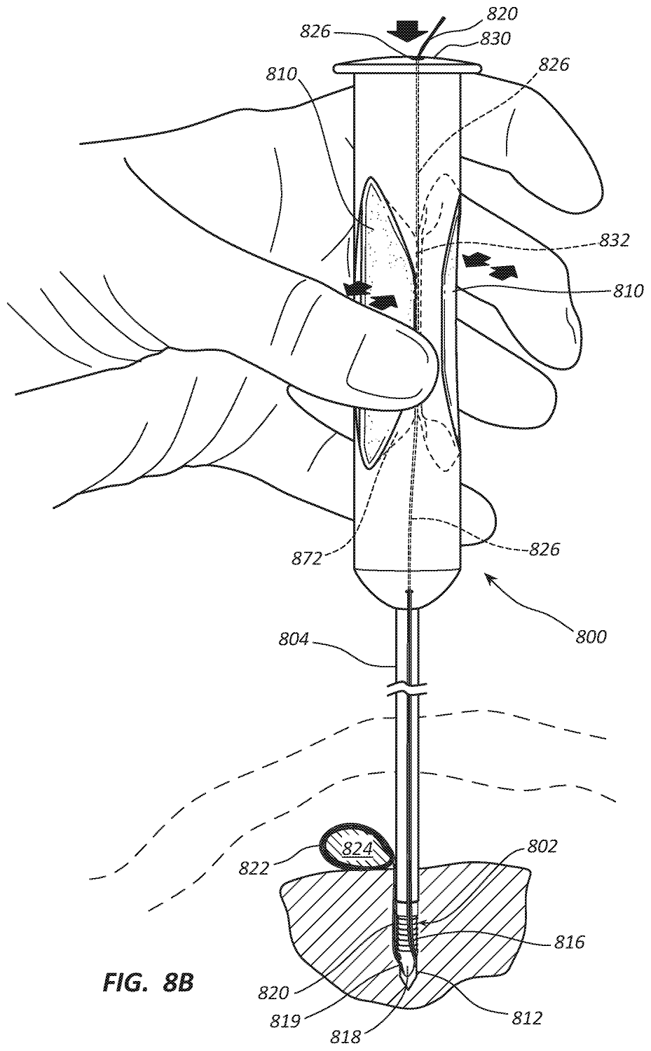

FIG. 8A-8B illustrate another embodiment of a driver apparatus 800 having a handle 808 equipped with at least one squeezable elastic suture brake 810. Driver apparatus 800 includes an elongate drive shaft 804 with a driver tip at the distal end to engage suture anchor 802. Handle 808 can be joined a proximal end of drive shaft 804. In some embodiments, the handle 808 may include only one brake 810, while in other embodiments it may be advantageous to incorporate two or more elastic brakes 810. When two or more brakes 810 are incorporated into handle 808, the brakes 810 can be manipulated independent of each another or in concert depending on user preference.

The handle 808 is adapted to receive free suture ends 820 of a suture 822 looped around connective tissue 824. During use, the free suture ends 820 can be threaded into an eyelet hole 819 through an eyelet portion 818 of a single stage push anchor 802 and then inserted through a suture passageway 826 in driver handle 808 that open through a distal side of handle 808 and exits through a distal side 830 of handle 808.

During use, the surgeon can engage the brake(s) 810 by squeezing the handle 808, which causes compression of elastic brake(s) 810 to apply friction to the free suture ends 820 within handle 808. Disengagement of brake 810 by releasing the squeezing force on handle 808 releases the braking force, thereby permitting longitudinal movement of free suture end 820 relative to handle 808. Elastic brake(s) 810 can be made of silicone, rubber, or other resilient or elastomeric material.

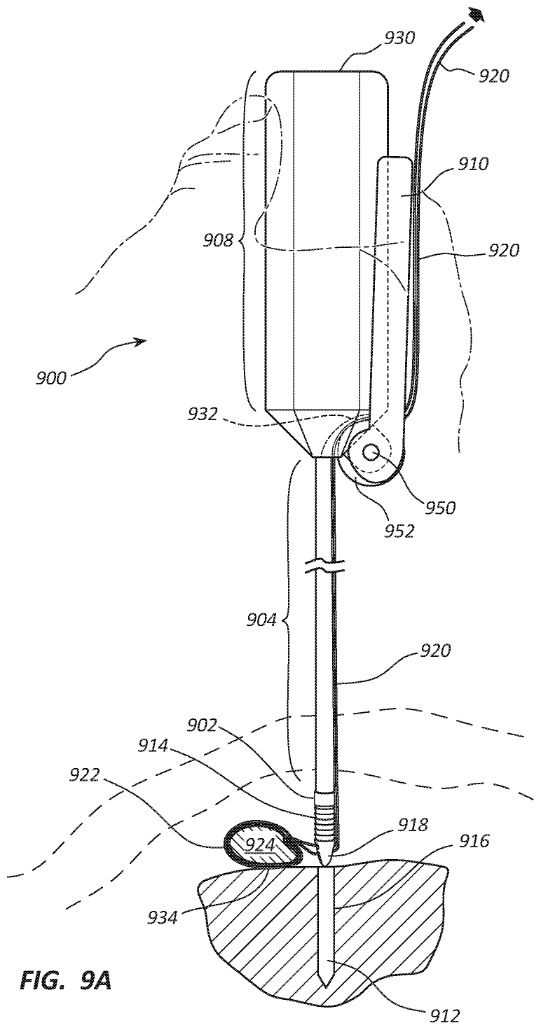

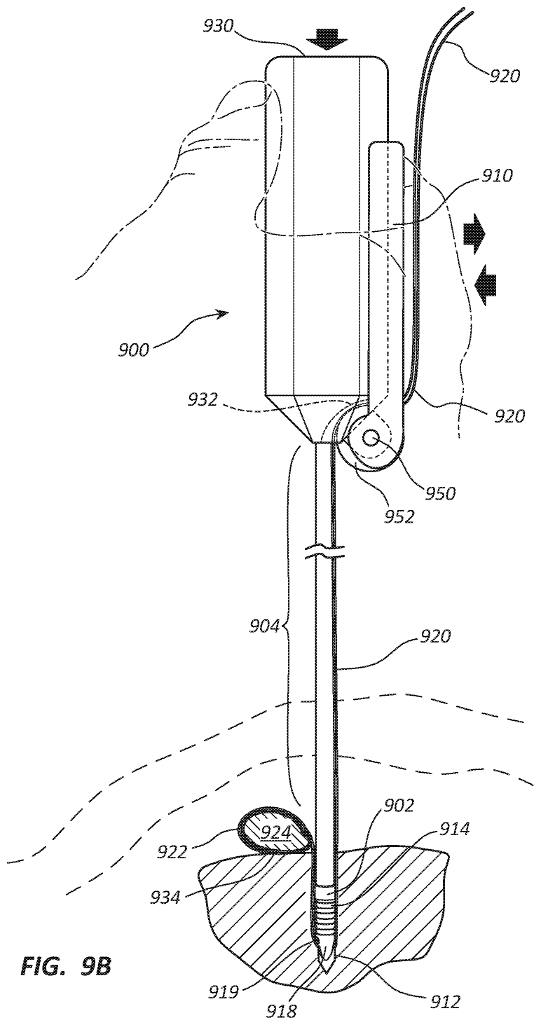

FIGS. 9A and 9B illustrate another embodiment of a driver apparatus 900 that is equipped with a manually actuatable suture brake lever 910 that includes a braking surface 932. Brake lever 910 is connected to a handle 908 by a pin 950, resulting in a pivot point. The brake lever 910 can be positioned outside of or partially within handle 908.

Suture brake lever 910 and braking surface 932 cooperate to press against free suture ends 920 of a suture 922 in order to prevent or restrict longitudinal movement of free suture ends 920 relative to handle 908. Brake lever 910 is typically deployed while in an upright position or parallel to handle 908, and can be opened or moved to a position transverse to handle 908 in order to allow the free suture end 920 to be fed through a gap between driver handle 908 and braking surface 932.

Embodiments of the invention, such as the examples disclosed herein, may be beneficial in a variety of respects. For example, and as will be apparent from the present disclosure, one or more embodiments of the invention may provide one or more advantageous and unexpected effects, in any combination, some examples of which are set forth below. It should be noted that such effects are neither intended, nor should be construed, to limit the scope of the claimed invention in any way. It should further be noted that nothing herein should be construed as constituting an essential or indispensable element of any invention or embodiment. Rather, various aspects of the disclosed embodiments may be combined in a variety of ways so as to define yet further embodiments. Such further embodiments are considered as being within the scope of this disclosure. As well, none of the embodiments embraced within the scope of this disclosure should be construed as resolving, or being limited to the resolution of, any particular problem(s). Nor should any such embodiments be construed to implement, or be limited to implementation of, any particular technical effect(s) or solution(s). Finally, it is not required that any embodiment implement any of the advantageous and unexpected effects disclosed herein.

1. A driver apparatus for use in driving a suture anchor into a bone tunnel while selectively adjusting suture tension in a connective tissue repair procedure, the driver apparatus comprising:
an elongated drive shaft comprising a driver tip at a distal end adapted to engage at least a portion of a suture anchor;
a driver handle associated with a proximal portion of drive shaft;
a suture brake associated with the driver handle; and
a passageway associated with the driver handle for receiving free suture ends of a suture attached to connective tissue,
wherein the suture brake is configurated so that when actuated the free suture ends are restrained from longitudinal movement relative to the driver handle and so that when released the free suture ends can move longitudinally relative to the driver handle.

2. The driver apparatus of claim 1, wherein the driver apparatus is adapted for use with a single stage push anchor, wherein the driver apparatus includes a single drive shaft fixedly attached to the driver handle.

3. A suture fixation system comprising the driver apparatus of claim 1 and a single stage push anchor adapted to be removably associated with the driver tip of the drive shaft.

4. The suture fixation system of claim 3, wherein the single stage push anchor includes an eyelet hole at a distal end adapted to receive one or more sutures therethrough and a recess at the proximal end adapted to engage the driver tip of the driver apparatus.

5. The driver apparatus of claim 1, wherein the driver apparatus is adapted for use with a two-stage push anchor having an eyelet portion and a barbed portion, wherein the drive shaft of the driver apparatus comprises:
a first stage drive shaft portion attached to the driver handle in a fixed longitudinal position at a proximal end having the driver tip at the distal end, wherein the driver tip is adapted to removably engage with the eyelet portion and push it into a bone tunnel in a first insertion stage without pushing in the barbed portion; and
a second stage drive shaft portion slidably associated with the first stage drive shaft portion, slidably received within the driver handle, and adapted to abut and drive the barbed portion of the two-stage push anchor into the bone tunnel toward the eyelet portion in a second insertion stage.

6. A suture fixation system comprising the driver apparatus of claim 5 and a two-stage push anchor adapted to be removably associated with the drive shaft and driver tip of the driver apparatus.

7. The suture fixation system of claim 6, the two-stage push anchor including:
an eyelet portion with an eyelet hole at a distal end adapted to receive one or more sutures therethrough and a recess at a proximal end adapted to engage the driver tip of the driver apparatus; and
a barbed portion detached from the eyelet portion and having a recess therethrough adapted to slidably associate the barbed portion with the first stage drive shaft portion and a proximal surface adapted to abut a distal end of the second stage drive shaft portion.

8. The driver apparatus of claim 1, wherein the driver apparatus is adapted for use with a two-stage threaded anchor having an eyelet portion and a threaded portion,
wherein the driver apparatus further comprises a driver knob fixedly attached to a proximal end of the drive shaft, the drive shaft being rotatably attached to the driver handle in a fixed longitudinal position,
wherein the driver tip is configured to rotatably engage the eyelet portion of the two-stage threaded anchor and push it into a bone tunnel in a first insertion stage,
the driver tip further including a longitudinal section with continuous cross-sectional shape configured to engage a correspondingly shaped recess through the threaded portion of the two-stage threaded anchor and cause rotational movement of the threaded portion in a second insertion stage by rotating the driver knob to drive the threaded portion into the bone tunnel toward the eyelet portion,
the longitudinal section of the driver tip with continuous cross-sectional shape permitting slidable longitudinal movement of the threaded portion along the longitudinal section as the threaded portion is advanced into the bone tunnel toward the eyelet portion.

9. A suture fixation system comprising the driver apparatus of claim 8 and a two-stage threaded anchor adapted to be removably associated with the drive shaft and driver tip of the driver apparatus.

10. The suture fixation system of claim 9, the two-stage threaded anchor including:
an eyelet portion with an eyelet hole at a distal end adapted to receive one or more sutures therethrough and a nipple at a proximal end adapted to be rotably received within a recess in the driver tip; and
a threaded portion detached from the eyelet portion and having a recess therethrough adapted to slidably associate the threaded portion with the driver tip and having a cross-sectional shape corresponding to the continuous cross-sectional shape of the longitudinal section of the driver tip.

11. The driver apparatus of claim 1, wherein the driver apparatus is adapted for use with a two-stage threaded anchor having an eyelet portion and a threaded portion,
wherein the driver apparatus further comprises a driver knob rotatably connected to the handle portion by a proximal portion of the drive shaft,
the drive shaft comprising a first stage drive shaft portion for pushing the eyelet portion into a bone tunnel in a first insertion stage and a second stage drive shaft portion that contacts and assists in driving the threaded portion into a bone tunnel in a second insertion stage,
the first stage drive shaft portion comprising:
a proximal end fixedly attached to the driver knob,
a proximal threaded section that threadably engages corresponding threads in an interior portion of the handle portion in a manner so that rotation of the driver knob relative to the handle portion causes the handle portion to move longitudinally relative to the driver knob, and
the driver tip at a distal end of the first stage drive shaft portion configured to rotatably engage the eyelet portion of the two-stage threaded anchor and push it into a bone tunnel in a first insertion stage,
the driver tip having a longitudinal section with continuous cross-sectional shape configured to engage a correspondingly shaped recess through the threaded portion of the two-stage threaded anchor and cause rotational movement of the threaded portion in a second insertion stage by rotating the driver knob to drive the threaded portion into the bone tunnel toward the eyelet portion,
the longitudinal section of the driver tip with continuous cross-sectional shape permitting slidable longitudinal movement of the threaded portion along the longitudinal section as the threaded portion is advanced into the bone tunnel toward the eyelet portion,
the second stage drive shaft portion being fixedly attached to and extending distally from the handle portion so as to move longitudinally together with the handle portion as the driver knob is rotated relative to the handle portion thereby remaining in contact with the threaded portion as it is driven into the bone tunnel in the second insertion stage.

12. A suture fixation system comprising the driver apparatus of claim 11 and a two-stage threaded anchor adapted to be removably associated with the drive shaft and driver tip of the driver apparatus.

13. The suture fixation system of claim 12, the two-stage threaded anchor including:
an eyelet portion with an eyelet hole at a distal end adapted to receive one or more sutures therethrough and a nipple at a proximal end adapted to be rotably received within a recess in a distal end of the first stage drive shaft portion; and
a threaded portion detached from the eyelet portion and comprising a recess therethrough adapted to slidably associate the threaded portion with the longitudinal section of continuous cross-sectional shape of the driver tip and having a cross-sectional shape corresponding to the continuous cross-sectional shape of the longitudinal section of the driver tip and a proximal surface adapted to abut a distal end of the second stage drive shaft portion.

14. The driver apparatus of claim 1, wherein the driver apparatus is adapted for use with a two-stage threaded anchor having an eyelet portion and a threaded portion,
wherein the driver apparatus further comprises a driver knob rotatably connected to the handle portion by a proximal portion of the drive shaft,
the drive shaft comprising a first stage drive shaft portion for pushing the eyelet portion into a bone tunnel in a first insertion stage and a second stage drive shaft portion that contacts and assists in driving the threaded portion into a bone tunnel in a second insertion stage,
the first stage drive shaft portion comprising:
a proximal end fixedly attached to the driver knob,
a proximal section that rotatably connects the driver knob to the handle portion in a fixed longitudinal position, and
the driver tip at a distal end of the first stage drive shaft portion configured to rotatably engage the eyelet portion of the two-stage threaded anchor and push it into a bone tunnel in a first insertion stage,
the driver tip having a longitudinal section with continuous cross-sectional shape configured to engage a correspondingly shaped recess through the threaded portion of the two-stage threaded anchor and cause rotational movement of the threaded portion in a second insertion stage by rotating the driver knob to drive the threaded portion into the bone tunnel toward the eyelet portion,
the longitudinal section of the driver tip with continuous cross-sectional shape permitting slidable longitudinal movement of the threaded portion along the longitudinal section as the threaded portion is advanced into the bone tunnel toward the eyelet portion,
the second stage drive shaft portion configured to rotate together with the driver knob and having a proximal threaded section positioned at least partially within and that threadably engages a corresponding threaded interior section of the handle portion in a manner so that rotation of the driver knob relative to the handle portion causes the second stage drive shaft portion to move longitudinally relative to the handle portion.

15. A suture fixation system comprising the driver apparatus of claim 14 and a two-stage threaded anchor adapted to be removably associated with the drive shaft and driver tip of the driver apparatus.

16. The suture fixation system of claim 15, the two-stage threaded anchor including:
an eyelet portion with an eyelet hole at a distal end adapted to receive one or more sutures therethrough and a nipple at a proximal end adapted to be rotably received within a recess in a distal end of the first stage drive shaft portion; and
a threaded portion detached from the eyelet portion and comprising a recess therethrough adapted to slidably associate the threaded portion with the longitudinal section of continuous cross-sectional shape of the driver tip and having a cross-sectional shape corresponding to the continuous cross-sectional shape of the longitudinal section of the driver tip and a proximal surface adapted to abut a distal end of the second stage drive shaft portion.

17. The driver apparatus of claim 1, wherein the driver apparatus is adapted for use with a knotless suture anchor with an internal locking mechanism.

18. A method for inserting a suture anchor into a bone tunnel in a connective tissue repair procedure to lock a suture within the bone tunnel at a fixed tension relative to connective tissue, the method comprising:
attaching a first portion of a suture to connective tissue and threading a second portion through an eyelet portion of the suture anchor;
positioning free ends of the suture in or adjacent to a handle of a driver apparatus comprising an elongated drive shaft having a driver tip adapted to engage the suture anchor, and a manually actuatable suture brake associated with the handle for selectively engaging and releasing the free suture ends to selectively prevent or permit longitudinal movement of the free suture ends relative to the handle;
positioning the eyelet portion of the suture anchor at a bone tunnel;
pulling the free ends of the suture to apply a suture tension;
inserting the eyelet portion into the bone tunnel to thereby increase a distance between the eyelet portion and the connective tissue;
selectively actuating and releasing the suture brake using the same hand gripping the driver handle to selectively prevent or permit longitudinal movement of the free suture ends relative to the handle and thereby increase or maintain the suture tension as the eyelet portion is progressively inserted in the bone tunnel.

19. The method of claim 18, wherein the suture anchor is single stage push anchor.

20. The method of claim 18, wherein the suture anchor is two-stage push anchor.

21. The method of claim 18, wherein the suture anchor is two-stage threaded anchor.
